(19) United States
(12) Patent Application Publication
(10) Pub. No.: US 2012/0100218 A1
(43) Pub. Date: Apr. 26, 2012

(54) Intralymphatic Chemotherapy Drug Carriers

(76) Inventors: Laird Forrest, Lawrence, KS (US); Mark Cohen, Overland Park, KS (US); Shuang Cai, Lawrence, KS (US)

(21) Appl. No.: 13/341,282

(22) Filed: Dec. 30, 2011

Related U.S. Application Data
(63) Continuation-in-part of application No. 12/363,302, filed on Jan. 30, 2009, now Pat. No. 8,088,412.
(60) Provisional application No. 61/024,837, filed on Jan. 30, 2008.

(51) Int. Cl. A61K 9/14; A61P 35/00; A61K 31/704; A61K 33/24; B82Y 5/00

(52) U.S. Cl. 424/489; 424/400; 514/34; 424/649; 977/773; 977/906

(57) ABSTRACT

A chemotherapeutic composition can be configured for subcutaneous administration for preferential intralymphatic accumulation while also providing a therapeutic systemic concentration that is not toxic. The composition can include a pharmaceutically acceptable carrier, and a nanoconjugate configured for preferential intralymphatic accumulation after subcutaneous administration. The nanoconjugate can include a nanocarrier configured for preferential intralymphatic accumulation after subcutaneous or interstitial administration, and a plurality of chemotherapeutic agents coupled to the nanocarrier. The nanoconjugate can have a dimension of about 10 nm to about 5 nm. Also, the nanoconjugate can be loaded with the chemotherapeutic agents from about 10% to about 50% w/w. The nanocarrier can be a hyaluronan polymer of about 3 kDa to about 50 kDa. Alternatively, the nanocarrier can be a dendrimer.

### CROSS-REFERENCE TO RELATED APPLICATIONS

This application is a continuation-in-part application of and claims priority to U.S. patent application Ser. No. 12/363,302 filed on Jan. 30, 2009; which is based on and claims priority to U.S. Provisional Application Ser. No. 61/024,837, filed Jan. 30, 2008, which are both incorporated herein by specific reference in their entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of grant R21 CA132033 and P20 RR015563 awarded by the National Institute of Health.

### BACKGROUND OF THE INVENTION

Cancer is a class of diseases in which a group of cells display uncontrolled growth and division beyond the normal limits, invasion into and destruction of adjacent tissues, and sometimes metastasis that spreads the cancer to other locations in the body via lymphatics or blood vessels. These malignant properties of cancers differentiate them from benign tumors, which are self-limited, do not invade, or metastasize. Most cancers form a tumor but some, like leukemia, do not. Cancer may affect people at all ages, even fetuses, but the risk for most varieties increases with age. Cancer causes about 13% of all deaths. According to the American Cancer Society, 7.6 million people died from cancer in the U.S. during 2007. Cancers can affect all animals.

Nearly all cancers are caused by abnormalities in the genetic material of the transformed cells. These abnormalities may be due to the effects of carcinogens, such as tobacco smoke, radiation, chemicals, or infectious agents. Other cancer-promoting genetic abnormalities may be randomly acquired through errors in DNA replication, or are inherited, and thus present in all cells from birth. The heritability of cancers are usually affected by complex interactions between carcinogens and the host's genome.

Diagnosis usually requires the histological examination of a tissue biopsy specimen by a pathologist, although the initial indication of malignancy can be symptoms or radiographic imaging abnormalities. Most cancers can be treated and some cured, depending on the specific type, location, and stage. Once diagnosed, cancer is usually treated with a combination of surgery, chemotherapy, and radiotherapy. As research develops, treatments are becoming more specific for different varieties of cancer. There has been significant progress in the development of targeted therapy drugs that act specifically on detectable molecular abnormalities in certain tumors, and which minimize damage to normal cells. The prognosis of cancer patients is most influenced by the type of cancer, as well as the stage, or extent of the disease. In addition, histological grading and the presence of specific molecular markers can also be useful in establishing prognosis, as well as in determining individual treatments.

Cisplatin (i.e., cis-diamminedichloroplatinum or CDDP) has become an important chemotherapeutic agent for many solid tumors. However, newer platinum drugs have been found to have fewer side effects, and such drugs may become important chemotherapeutic agents. One drawback to cisplatin as well as other chemotherapeutics or potential chemotherapeutics is significant toxicity.

Since organ toxicities hamper chemotherapy, oncologists have developed procedures to confine chemotherapy to the diseased areas by temporarily isolating the affected tissues or organs from the systemic circulation and perfusing them with the chemotherapeutic. For example, intra-arterial percutaneous pelvic perfusion of high-dose chemotherapeutic can provide a therapeutic advantage in advanced uterine cervical carcinoma with low side effects. However, these treatments are highly invasive and require specialized skill's and equipment usually restricted to large medical research centers. In addition, tissue isolation is not possible in many cases, including locally advanced breast cancer that has significant invasion into lymphatic tissues.

Treatment of locally advanced breast cancer may be improved if chemotherapy were concentrated to the breast lymphatics, while maintaining adequate systemic levels for treatment of distant metastases. Neoadjuvant systemic chemotherapy is standard care for locally advanced breast cancer (“LABC”), but after treatment cancer typically spreads first via the lymphatics with little stroma invasion before becoming a systemic disease. Surgical treatment for early stage breast cancer involves resection of the primary tumor along with the draining sentinel lymph node and further lymphatic resection if warranted. However, this procedure may miss nanoscopic metastases in the lymph nodes if full immunohistochemical analysis is not routinely performed on sentinel node specimens, which is estimated to double the risk of relapse (compared to truly node negative cases). Localized radiation to the breast and lymphatics along with systemic chemotherapy reduce the risks of relapse but these treatments cause extensive damage to healthy tissues.

Regardless of their origin, many cancers metastasize by using the lymphatic system (e.g., breast, ovarian, melanoma). The lymphatics are the body's drainage system, clearing waste from the tissues, and metastatic cancers follow this drainage to “seed” first in the local lymphatics. Surgery and chemotherapy can destroy many of these early metastases, but with great morbidity to the patient (e.g., toxicity side effects and painful lymphedema). Thus, it would be beneficial to have a chemotherapeutic that avoids these side effects by delivering chemotherapy directly to the tumor tissue in early cancers. Also, it would be advantageous for a chemotherapeutic to be preferentially directed into the lymphatics, and thereby avoiding side effects on normal cells elsewhere in the body, destroying the “seed's” that can cause recurrence after surgery and whole-body chemotherapy.

### BRIEF SUMMARY OF THE INVENTION

In one embodiment, the present invention includes a chemotherapeutic composition configured for local administration by percutaneous injection. The composition can include a pharmaceutically acceptable carrier; and a nanoconjugate configured for preferential intralymphatic accumulation after percutaneous (where percutaneous refers to subcutaneous, intradermal, peritumoral, submucosal, or transdermal) administration.

In one embodiment, the present invention includes a nanoconjugate comprising: a nanocarrier configured for preferential intralymphatic accumulation after percutaneous or interstitial administration; and a plurality of chemotherapeutic agents coupled to the nanocarrier. The nanoconjugate may have a dimension of about 10 nm to about 80 nm. Also, the nanoconjugate can preferably be loaded with the chemotherapeutic agents from about 10% to about 50% w/w (e.g. about 10, 15, 20, 25, 30, 35, 40, 45, or 50% w/w, or some range therebetween). The nanocarrier can preferably be a hyaluronan polymer of about 20 kDa to about 150 kDa (e.g. about 20, 25, 30, 35, 40, 45, 50, 55, 60, 65, 70, 75, 80, 85, 90, 95, 100, 105, 110, 115, 120, 125, 130, 135, 140, 145 or 150 kDa, or some range therebetween), although hyaluronan polymers up to about 200 kDa are suitable. Higher molecular weight hyaluronan polymers may disadvantages because of increased viscosity. Alternatively, the nanocarrier can be a dendrimer. These chemotherapeutic agents may be categorized by their mechanism of action into, for example, following groups: anti-metabolites/anti-cancer agents, such as pyrimidine analogs (5-fluorouracil, floxuridine, capecitabine, gemcitabine, and cytarabine) and purine analogs, folate antagonists and related inhibitors (mercaptopurine, thioguanine, pentostatin, and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, mechlorethamine, mitomycin, mitoxantrone, nitrosourea, paclitaxel, plicamycin, procarbazine, teniposide, triethylenethiophosphoramide, and etoposide (“VP16”)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (“BCNU”) and analogs, streptozocin), trazenes-dacarbazinine (“DTIC”); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts, and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (“FK-506”), sirolimus (rapamycin), azathioprine, mycophenolate mofetil); anti-angiogenic compounds (TNP-470, genistein) and growth factor inhibitors (vascular endothelial growth factor (“VEGF”) inhibitors, fibroblast growth factor (“FGF”) inhibitors, epidermal growth factor (“EGF”) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (“CPT-11”) and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; chromatin disruptors; and combinations thereof. In one aspect, the chemotherapeutic agents are selected from cisplatin, other platinum chemotherapeutic drugs, melphalan, withaferin A, mytomycin C, doxorubicin, epirubicin, docetaxel, daunorubicin, combinations thereof, and the like. Most preferred combinations involve nanoconjugates of cisplatin, doxorubicin, and/or docetaxel. For example, the combination may comprise (1) HA-cisplatin+HA-doxorubicin; (2) HA-cisplatin+HA-docetaxel; (3) HA-doxorubicin HA-docetaxel; or (4) HA-cisplatin HA-doxorubicin+HA-docetaxel.

For combination therapy or coadministration therapy, at least two active compounds in effective amounts are used to treat cancer as otherwise described herein at the same time. Although the combination therapy or coadministration preferably includes the administration of two active compounds to the patient at the same time, it is not necessary that the compounds be administered to the patient at the same time, although effective amounts of the individual compounds will be present in the patient at the same time. Further, the combination therapy may include nanoconjugate compositions containing one or more of the chemotherapeutic agents (e.g., nanocarriers coupled to doxorubicin and nanocarriers coupled cisplatin mixed together). The nanocarriers forming the nanoconjugate compositions may be coupled to one or more chemotherapeutic agents (e.g., nanocarriers coupled to both doxorubicin and cisplatin or nanocarriers coupled to doxorubicin, cisplatin, and docetaxel).

In one embodiment, compositions contain between about 40% and 90% of the maximum tolerated dose of the chemotherapeutic agent(s). The chemotherapeutic agents are preferably selected from the group consisting of cisplatin, doxorubicin, and docetaxel, although various combinations of the chemotherapeutic agents described herein are contemplated. In one aspect, the nanoconjugate compositions comprise about 10% to 100% (e.g., about 10, 15, 20, 25, 30, 35, 40, 45, 50, 55, 60, 65, 70, 75, 80, 85, 90, 95, or 100%) of the maximum tolerated dose of doxorubicin. In one aspect, the nanoconjugate compositions comprise about 10% to 100% (e.g., about 10, 15, 20, 25, 30, 35, 40, 45, 50, 55, 60, 65, 70, 75, 80, 85, 90, 95, or 100%) of the maximum tolerated close of cisplatin. In another aspect, the nanoconjugate compositions comprise about 10% to 100% (e.g., about 10, 15, 20, 25, 30, 35, 40, 45, 50, 55, 60, 65, 70, 75, 80, 85, 90, 95, or 100%) of the maximum tolerated dose of doxorubicin and comprise about 10% to 100% (e.g., about 10, 15, 20, 25, 30, 35, 40, 45, 50, 55, 60, 65, 70, 75, 80, 85, 90, 95, or 100%) of the maximum tolerated dose of cisplatin. In still another aspect, the nanoconjugate compositions comprise about 10% to 100% (e.g., about 10, 15, 20, 25, 30, 35, 40, 45, 50, 55, 60, 65, 70, 75, 80, 85, 90, 95, or 100%) of the maximum tolerated dose of doxorubicin and comprise about 1.0% to 100% (e.g., about 10, 15, 20, 25, 30, 35, 40, 45, 50, 55, 60, 65, 70, 75, 80, 85, 90, 95, or 100%) of the maximum tolerated dose of cisplatin and also comprise about 10% to 100% (e.g., about 10, 15, 20, 25, 30, 35, 40, 45, 50, 55, 60, 65, 70, 75, 80, 85, 90, 95, or 100%) of the maximum tolerated dose of docetaxel.

In one embodiment, the chemotherapeutic agents are coupled to the nanocarrier via a biodegradable linker. For example, the biodegradable linker is acid-labile or degradable.

In one embodiment, the chemotherapeutic composition and/or nanoconjugate is substantially devoid of PEG, HPMA, polyglutames, and/or silver.

In one embodiment, the chemotherapeutic agent is present in a therapeutically effective amount so as to provide a higher lymphatic AUC and a lower plasma compared to standard intravenous administration of the chemotherapeutic agent.

In one embodiment, the present invention includes a method for treating and/or inhibiting cancer. Such a method can include percutaneously administering a composition having a pharmaceutically acceptable carrier, and a nanoconjugate configured for preferential intralymphatic accumulation after subcutaneous administration. The nanoconjugate can be any embodiment as described herein. Further, the composition may be co-administered with one or more other active agents, such as the chemotherapeutics described herein.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, amore particular description of the invention will be rendered by reference to specific embodiments thereof which are illustrated in the appended drawings. It is appreciated that these drawings depict only illustrated embodiments of the invention and are therefore not to be considered limiting of its scope. The invention will be, described and explained with additional specificity and detail through the use of the accompanying drawings in which:

FIGS. 1A-1B include graphs that illustrate the tissue and plasma concentration of chemotherapeutic agents. FIG. 1A is a graph that shows the tissue concentration of platinum in ipsilateral (right) axillary nodes and contralateral (left) axillary nodes after intravenous injection of cisplatin or subcutaneous injection of HA-Cisplatin (3.3 mg/kg cisplatin-basis) into the right mammary fatpad. FIG. 1B is a concentration vs. time pharmacokinetics graph that shows the plasma concentration of cisplatin after either intravenous injection of cisplatin (3.3 mg/kg) or subcutaneous injection of HA-cisplatin (3.3 mg/kg) in the right mammary fatpad.

FIGS. 2A-2B include graphs that illustrate the concentration of creatinine in urine over time after a single dose administration of subcutaneous HA-cisplatin in the right mammary fatpad. FIG. 2A is a graph that shows the urine creatinine concentration of animal's that received 3.3 mg/kg HA-cisplatin with or without silver. FIG. 2B is a graph that shows the urine creatinine concentration of animals that received 1.0 mg/kg HA-cisplatin with or without silver. In FIG. 2A, lower urine creatinine is a sign of renal damage as seen with the high close samples containing silver, whereas in FIG. 2B, there was no significant difference between the two formulations at low doses.

FIGS. 3A-3F are images of kidney tissue 30 days post single injection with drug compound and stained with hematoxylin and eosin. FIG. 3A shows that animals receiving subcutaneous HA had normal tissues (control). FIG. 3B shows that animals receiving 3.3 mg/kg intravenous cisplatin had degenerative changes such as pyknotic nuclei in corticomedullary tubular cells. FIG. 3C shows that animals receiving subcutaneous 3.3 mg/kg HA-cisplatin without silver had fairly normal appearance except for minor foci of tubular cell necrosis at the corticomedullary junction. FIG. 3D shows that animals receiving 1.0 mg/kg subcutaneous HA-cisplatin with silver had widely spread pyknotic nuclei in medullary tubular epithelial cells. FIG. 3E shows that animals receiving 1.0 mg/kg intravenous cisplatin had pyknotic nuclei in medullary tubular epithelial cells, increases in dark purple staining suggesting nuclear staining and spread apoptosis. FIG. 3F shows that animals receiving 1.0 mg/kg subcutaneous HA-cisplatin had normal appearance except for minimal renal tubular cell swelling and degeneration.

FIGS. 4A-4F are images of liver tissue 30 days post single injection with drug and stained with H&E. FIG. 4A shows that animals receiving subcutaneous HA had normal tissue (control). FIG. 4B shows that animals receiving 3.3 mg/kg cisplatin had moderate necrosis.

FIG. 4C shows that animals receiving 3.3 mg/kg subcutaneous HA-cisplatin had fairly normal appearance except for very mild degeneration. FIG. 4D shows that animals receiving 1.0 mg/kg HA-cisplatin with silver had fairly normal appearance except for very mild degeneration. FIG. 4E shows that animals receiving 1.0 mg/kg intravenous cisplatin had fairly normal appearance except for very mild degeneration. FIG. 4F shows that animals receiving 1.0 mg/kg subcutaneous HA-cisplatin had normal appearance.

FIGS. 5A-5F are images of brain tissue 30 days post single injection with drug and stained with H&E. Animals receiving subcutaneous injection of HA (control) and all study compounds (e.g., intravenous cisplatin 3.3 mg/kg, subcutaneous HA-cisplatin 3.3 mg/kg, subcutaneous HA-cisplatin with Ag 1 mg/kg, intravenous cisplatin 1 mg/kg, subcutaneous HA-cisplatin 1 mg/kg) had normal findings.

FIGS. 6A-6F are images of lymphoid tissue 30 days post injection and stained with H&E. Animals receiving subcutaneous injection of HA (control) and all study compounds (e.g., intravenous cisplatin 3.3 mg/kg, subcutaneous HA-cisplatin 3.3 mg/kg, subcutaneous HA-cisplatin with Ag 1 mg/kg, intravenous cisplatin 1 mg/kg, subcutaneous HA-cisplatin 1 mg/kg) had normal findings.

FIGS. 7A-7D are images of the underlying tissue at injection site 30 days post injection. Animals receiving subcutaneous injection of HA (control) and all study compounds (e.g., subcutaneous HA-cisplatin 3.3 mg/kg, subcutaneous HA-cisplatin with Ag 1 mg/kg, HA-cisplatin 1 mg/kg) had normal findings.

FIGS. 8A-8H are graphs illustrating tissue concentrations of platinum after intravenous injection of cisplatin (3.3 mg/kg cisplatin basis) or subcutaneous injection of HA-cisplatin (3.3 mg/kg cisplatin basis) into right mammary fatpad. FIG. 8A is for the bladder.

FIG. 8B is for the brain. FIG. 8C is for the heart. FIG. 8D is for the kidney. FIG. 8E is for the liver. FIG. 8F is for the lungs. FIG. 8G is for the muscle. FIG. 8H is for the spleen.

FIG. 9 is a schematic representation of the synthesis of an intralymphatic chemotherapeutic and its function in chemotherapy.

FIGS. 10A-10B are graphs that illustrate the total amount of agent after subcutaneous injection. FIG. 10A shows the tissue concentration of cisplatin in right axilla lymph nodes (“RLN”) and left axilla lymph nodes (“LLN”) after subcutaneous injection of cisplatin or cisplatin-HA (3.3 mg/kg cisplatin basis) into right mammary fatpad. FIG. 10B shows the plasma concentration of cisplatin under the same procedure. Of note, serum Cₘₐₓ for intravenous cisplatin is over 4 micrograms/mL whereas for HA-cisplatin it is less than 3 micrograms/mL. High Cₘₐₓ with cisplatin has been directly linked with ototoxicity, nephrotoxicity, and peripheral neuropathy associated with this drug. This data supports that HA-cisplatin may be less toxic than intravenous cisplatin.

FIGS. 11A-11H are tissue concentration graphs for various tissue (e.g., FIG. 11A is bladder, FIG. 11B is brain, FIG. 11C is heart, FIG. 11D is kidney, FIG. 11E is liver; FIG. 11F is lungs, FIG. 11G is muscle, and FIG. 11H is spleen) concentrations of cisplatin after subcutaneous injection of cisplatin-HA (10 mg/kg cisplatin basis) into the right mammary fatpad.

FIG. 12 is a graph that illustrates cell viability through the inhibition of human cancer cell growth by cisplatin and cisplatin-HA after 72 hours. As a note, HA by itself showed no toxicity over the examined concentrations (up to 10 mg/mL, data not shown). This graph demonstrates that conjugating HA to CDDP did not adversely effect the anticancer effect of cisplatin in vitro as all of the cell lines demonstrated similar IC50 levels.

FIGS. 13A-13C are photographs showing the localization of the intralymphatic carrier after subcutaneous injection in nude mice bearing MDA-MB-468 breast lymphatic tumors expressing green fluorescent protein (“GFP”) FIG. 13A shows the breast lymphatic tumor 4 at the time that the mice were subcutaneously injected with Texas Red-HA 6 in the left mammary fat pad. After 5 hours and 18 hours (FIG. 13B and FIG. 13C, respectively), the photographs show that significant HA localized in the draining nodes and co-located with the tumor (GFP-channel in green in color and marked with 4, Texas Red channel in red and marked with 6, the blue arrow 2 is the injection site).

FIGS. 14A-14C are schematic diagrams of the synthesis of nanoconjugates.

FIG. 15A is a schematic diagram illustrating the synthesis of a dendrimer.

FIG. 15B is a schematic diagram illustrating the conjugation of targeting agents to nanoconjugates.

FIGS. 17A-17B show that tumor growth was delayed by HA-cisplatin treatment for 5 weeks compared to negative control group and 2 weeks compared to conventional cisplatin treatment.

FIG. 18 shows the release of doxorubicin as a function of pH. The release half-life was found to be 167 hours at pH 7.4, 107 at pH 6.0, and 45 at pH 5.0.

FIG. 19 shows the tumor growth was halted by nanocarrier-DOX treatment after two weekly closes at 3rd and 5th week, a significant improvement in efficacy compared to standard intravenous doxorubicin (purple line).

FIG. 20A illustrates a phosphoester-HA.

FIG. 20B is a schematic diagram illustrating the synthesis of nanoconjugates with phosphoester-HA.

FIG. 21A is a graph that illustrates the in vivo efficacy of subcutaneous HA-cisplatin administration.

FIGS. 21B-21C are graphs that illustrate standard cell viability vs. drug concentration curves by MTS assay comparing in vitro antiproliferative properties of standard CDDP formulation (FIG. 21B) with HA-Cisplatin (FIG. 21C) against two human head and neck squamous carcinoma cell lines (JMAR and MDA-1986). Of note, the IC50 levels were very similar with both drugs indicating that HA conjugation again did not adversely effect the anticancer activity of CDDP in vitro.

FIGS. 22A-22F are photographs showing the distribution of HA-doxorubicin after a single injection in the right mammary fat pad of a rat. Doxorubicin has innate fluorescence and the distribution and longevity of the drug-carrier conjugate can been well observed in this timed evaluation. Of note the bulk of drug-carrier is transported to the axillary lymph nodes where is slowly releases drug over a 9 day interval with still some residual activity even after 9 days. The oval marks the injection site in the breast and the darkest concentration (red) is in the axilla.

FIG. 23 is a graph showing tumor response even of a single late term peritumoral HA-Doxorubicin treatment in a considerably advanced breast cancer tumor in vivo.

FIGS. 24A-24E are photographs showing in vivo trafficking of HA-doxorubicin as visualized on a Maestro multichannel fluorescent imaging system. There is nice uptake of drug and carrier into the locoregional tissues and lymph nodes of the rat breast, which stays well in the lymphatic's even 4 days post-injection.

FIG. 25 shows the comparison of breast tumor volumes with treatment. The graph shows a composite curve of the animals in control and four treatment groups (HA-Dox-Cis 50, HA-Dox-Cis 75, Dox-Cis 50 and Dox-Cis 75), N=8 for each group. The control curve is a composite curve of HA-carrier s.q. injection and 1×PBS systemic injection (N=4 for each). Note, there is delay in tumor growth with standard Dox-Cis treatment, however, progressive disease does still occur whereas there were significantly more complete responders in the HA treatment groups, which was durable.

FIG. 26 illustrates the efficacy of the treatment by histologic confirmation. FIG. 26A shows the whole body image of mouse treated with HA-Dox-Cis 50 at week 12. The arrow denotes no clinical evidence of residual tumor and normal appearing skin at the injection site. FIG. 26B shows the whole body image of mouse treated with Dox-Cis 50 at week 12. Here, the arrow notes progressive tumor growth with ulceration following treatment. FIG. 26C is a hematoxylin and eosin stained histologic image at 7.8× magnification of mouse from image FIG. 26A. The arrows denote skin, normal breast tissue surrounding injection site with polymorphonuclear leukocyte infiltration and associated fibrosis. Of note, there is no histologic evidence of tumor present, indicating a complete pathological response. FIG. 26D is a hematoxylin and eosin stained histologic image at 5.4× magnification of mouse treated with Dox-Cis 50 demonstrating a partial response clinically. Arrows denote skin, histological presence of tumor with associated central necrosis.

FIG. 27A summarizes the clinical evaluation of animal Toxicity by weight changes. Of note, there was a 23% weight loss observed in the Dox-Cis 75 group compared no durable weight loss in the HA-Dox-Cis groups, which was statistically significant (p<0.001).

FIG. 27B shows the Kaplan-Meier survival curves by group. Note that both HA-Dox-Cis groups had 100% survival throughout the study, which was superior to the Dox-Cis groups. N=6 for each group as 2 animals were euthanized immediately following treatment for histology. Note, controls were all euthanized by week 7 due to advanced tumor volumes and deteriorating body condition from progressive disease per established animal protocol endpoints, and therefore were not included in FIGS. 27A and 27B.

FIG. 28 is the synthesis scheme for preparing HA-docetaxel in accordance with the present invention.

FIG. 29 shows the release kinetics of the HA-docetaxel nanoconjugate.

### DETAILED DESCRIPTION OF THE PREFERRED EMBODIMENTS

Generally, the present invention is related to new chemotherapeutic agents, pharmaceutical compositions having the chemotherapeutic agents, methods of making the chemotherapeutic agents, and methods of administering the chemotherapeutic agents in a manner for preferential accumulation in the lymphatic system. The chemotherapeutic agent includes a nanocarrier that is preferentially routed into the lymphatic system upon administration into substantially any interstitial site within a body, such as adjacent to a tumor or subcutaneously, but is not systemically administered. That is, the formulation is not administered via intravenous administration. As such, the chemotherapeutic agent preferentially targets any sized tumors, cancerous cells, or other malignancies within the lymphatic system. Such an approach can effectively inhibit the spread of cancerous cells from an initial cancer to another part of the body.

The design of the chemotherapeutic agent, which is a nanoconjugate, allows for translocation from the site of injection/administration through the lymphatic system so as to not produce elevated peak systemic concentrations that are toxic. Traditional administration routes, such as intravenous, produce high peak systemic concentrations usually via first-pass pharmacokinetics of the drug that are toxic and should be avoided. Therefore, the chemotherapeutic agent preferentially translocates into the lymphatic system, and can treat cancerous cells that are in the lymphatic system, which include cells that may be residing in a lymph node. This mode of translocation follows the route many cancerous cells follow when initially spreading from their primary focus, and thereby can be used to treat or inhibit the spread of cancerous cells through the locoregional tissues.

The chemotherapeutic agent includes a nanocarrier that is optimized in size and composition to preferentially be translocated into the lymphatic system rather than spread and concentrate systemically. It has been found that hyaluronan polymeric carriers and some dendritic carriers, such as those described herein, have such as selective translocation characteristic into the lymphatic system. For example, the chemotherapeutic agent can be deposited adjacent to a skin cancer and enter the lymphatic system similar to the cancerous cells.

Cisplatin is one of the most widely used chemotherapy agents for solid tumors; however, its toxicity and resistance severely limits its dose and use in many patients. Penetration of systemic cisplatin into the lymphatics may be poor (less than 1-5% of drug injected), and alternatives treatments for localized cancers (e.g., surgical removal or radiation) can lead to serious side effects such as infections and lymphedema. As such, a method of treating or inhibiting the accumulation of cancerous cells in the lymphatics is desirable, and can now be accomplished through the subcutaneous or interstitial administration of a nanoconjugate that preferentially translocates to the loco-regional tissues and lymphatics. The nanoconjugate can include a nanocarrier of a FDA-approved biocompatible carrier coupled to a chemotherapeutic drug (e.g., cisplatin). The nanoconjugate can also decrease systemic toxicities compared to traditional intravenous therapy with cisplatin. This allows for the treatment, inhibition, and/or prevention of many cisplatin responsive cancers, such as breast, non-small cell lung, ovarian, and head and neck squamous cell carcinoma and other cisplatin-sensitive tumors, as the lymphatics are involved in early to late stages of the spread of the disease. Cisplatin is also referred to herein as CDDP and Pt, such as in the examples and figures.

Previously, targeting and nanocarrier strategies have been reported to increase the dosage of cisplatin reaching tumor-bearing tissues, while sparing normal tissues from toxic doses. These technologies can be categorized into untargeted or passively targeted carriers (e.g., non-receptor) and actively targeted carriers (e.g., antibodies). Among the untargeted carriers such as polymeric micelles formulations (e.g., NC-6004) have demonstrated reduced nonspecific toxicities in preclinical studies and have progressed to early clinical trials. In phase I trials, an untargeted linear polymer conjugate of N-(2-hydroxypropyl)methacrylamide (“HMPA”) and platinum (e.g., AP5280) had demonstrated higher sustained plasma levels of platinum in humans with minimum toxicity compared to intravenous cisplatin. These untargeted carriers rely on the enhanced permeability and retention effect (“EPR”) to improve tumor accumulation of the drug, but in tumors that are not highly vascularized, the EPR effect is greatly reduced, and untargeted nanocarriers have less advantages. Accordingly, passive targeting is not effective in the treatment of tumors with low vascularity, such cancerous cells that can be found in the lymphatic system.

Now, with the present invention cancerous cells that are not vascularized, such as those found within the lymphatic system, can be treated or inhibited by selectively accumulating a chemotherapeutic agent in the loco-regional lymphatics of the tumor via a subcutaneous injection at the site of the tumor, allowing the chemotherapeutic drug to be delivered along the lymphatic pathway where tumors are most likely to initially metastasize. This can be performed with a nanoconjugate of a biocompatible nanocarrier and a chemotherapeutic drug, where the nanocarrier provides for site selective accumulation of a drug that is difficult to deliver without a carrier.

The subcutaneous administration of a nanoconjugate that can accumulate in intralymphatic tissues is beneficial for treating many cancers, such as breast cancers. For example, breast cancers typically spread to regional lymph nodes once they disseminate from the primary tumor, thus adequate evaluation and treatment of the axillary lymph nodes is important in early stage disease. One significant problem with current therapy is the side effects chemotherapy agents create systemically either alone or in combination. The nanoconjugates of the present invention are advantageous because they, surprisingly and unexpectedly, can accumulate in the cancerous cells that are present in the lymphatic system and/or intralymphatic tissues and thereby act on lymphatic metastases without undesirable systemic toxicities. The nanoconjugates can be subcutaneously administered for accumulation in intralymphatic tissue. As such, the nanoconjugates can be used to treat breast cancer to preferentially treat at-risk regional lymph nodes and avoid systemic toxicities.

The present invention can include a nanoconjugate of the polysaccharide hyaluronan (“HA”) with a chemotherapeutic drug (e.g., cisplatin or other platinum), pharmaceutical compositions and methods related to the same, especially those related to subcutaneous injection for achieving therapeutic intralymphatic localization and non-toxic systemic concentrations. The HA nanoconjugate is formulated with a molecular weight/size of HA that is effective in concentrating cisplatin to the breast lymphatics, and reduce peak plasma concentrations that are toxic. While cisplatin has been used herein as a representative chemotherapeutic drug, other drugs that are shown to be effective in chemotherapy can be conjugated to the nanoconjugates of the present invention. Also, HA is used as a representative nanocarrier; however, other nanocarriers with the same or similar physiological delivery profiles and properties, such as dendrimers, can be used. Cisplatin was used and described herein because of the ease of determining platinum deposition in organs, tissues, and lymphatics by atomic absorption spectroscopy. As such cisplatin deposition after administration is representative of other chemotherapeutic drugs that can: be conjugated to the nanocarrier.

In one embodiment, the chemotherapeutic drug is cisplatin, which has been shown to be an excellent anticancer agent for many solid tumors, but the standard formulation of cisplatin has been shown to have significant systemic toxicity. Now, the nanoconjugate of cisplatin has been shown to capable of being administered subcutaneously as a loco-regional delivery system to increase platinum levels in the lymphatics, where early metastasis is most likely to occur, while reducing systemic toxicities. The cisplatin nanoconjugate surprisingly and unexpectedly, also can provide suitable systemic concentrations that are therapeutically effective without significant systemic toxicity. As demonstrated in the figures in this application, HA-cisplatin is able to provide serum and systemic AUCs which are therapeutically effective but without the toxic high. Cmax levels of standard intravenous CDDP. The combination of being capable of being delivered locally via subcutaneous administration (e.g., proximal the tumor) for therapeutically effective intralymphatic accumulation and systemic concentrations and having less toxic sustained release characteristics make it more advantageous than standard CDDP.

For example, cisplatin can be conjugated to a biocompatible polymer such as hyaluronan, with a conjugation degree of approximately 20 w/w %. The nanoconjugates can be delivered via subcutaneous injection (e.g., into the breast tissue of rats) for therapeutically effective intralymphatic accumulation and systemic concentrations. The HA-cisplatin nanoconjugate demonstrated antiproliferative efficacy similar to standard cisplatin formulations in human breast cancer in vitro. The nanoconjugate increased the plasma area-under-the-curve (“AUC”) by 2.7-fold compared to normal cisplatin, but with a reduced peak plasma level (“Cₘₐₓ”) which is beneficial for reducing systemic toxicity. The nanoconjugate increased the ipsilateral lymph node AUC by 3.8-fold compared to cisplatin. Pathology studies of animals receiving the nanoconjugate treatment showed normal appearance of brain and lymph nodes, with less necrosis and inflammation in the kidneys and liver compared to intravenous administered cisplatin. Thus, the nanoconjugate demonstrates that intralymphatic drug delivery with hyaluronan-based chemotherapeutic drugs may allow lower drug dosing, levels with less toxicity than intravenous therapies while providing a “boost” close of the chemotherapeutic drug in the loco-regional tissue basin where tumor burden is highest.

Generally, the nanocarrier may be conjugated to peptides, antibodies (both monoclonal and polyclonal), interferon, other nitrogen mustard class drugs besides melphalan including chlorambucil, amiodarone, topotecan, withaferin A, HSP90 inhibitors including 17-AAG, VEGF inhibitors, histone deacetylase inhibitors, and any of the taxanes including taxol, paclitaxel, docetaxel, and the like. Some examples of drugs that can be conjugated to the nanoconjugate include cisplatin, other platinum drugs, melphalan, mytomycin C, doxorubicin, epirubicin, docetaxel, daunorubicin, chlorambucil, 517U, paclitaxel, vincristine, Her2 antibodies and peptides, EGFR antibodies and peptides, rapamycin, mTOR inhibitors, withaferin A, HDAC inhibitors, SAHA, Hsp90 inhibitors, 17-AAG, and 17-DMAG.

In one embodiment, the nanocarrier is a hyaluronan polymer, which is a highly biocompatible polymer that has now been found to follow lymphatic drainage from the interstitial spaces, such as from subcutaneous administration. The nanoconjugates of HA and cisplatin can be formed by non-covalent conjugation or through biodegradable bonds, such as ester or hydrazine bonds. The nanoconjugates can be injected subcutaneously anywhere in the body. Examples include injection into the upper mammary fat pad of female subjects for treatment of breast cancer.

Hyaluronan polymer is a polysaccharide, of alternating D-glucuronic acid and N-acetyl D-glucosamine, found in the connective tissues of the body and cleared primarily by the lymphatic system (12 to 72 hours turnover half-life). After entering the lymphatic vessel, HA is transported to lymph nodes where it is catabolized by receptor-mediated endocytosis and lysosomal degradation. Several studies have correlated increased HA synthesis and uptake with cancer progression and metastatic potential. Breast cancer cells are known to have greater uptake of HA than normal tissues, requiring HA for high P-glycoprotein expression, the primary contributor to multi-drug resistance. Furthermore, invasive breast cancer cells overexpress CD44, the primary receptor for HA, and are dependent on high concentrations of CD44 internalized HA for proliferation. Thus, chemotherapeutic drug nanoconjugates with HA may be efficacious against lymphatic metastases.

Accordingly, HA-drug nanoconjugates can be directed to the lymphatic system and accumulate in lymph nodes by binding to CD-44 receptors on the lymph node surface and cancer cells where the CD-44 receptors are overexpressed. Hyaluronan is also a ligand for CD44 receptor and is cleared primarily by the lymphatic system where it is catabolized in the nodes by CD44 receptor-mediated endocytosis followed by lysosomal degradation. This allows the drug in the nanoconjugate to be delivered to the site of initial tumor spread, concentrating its effects in the lymph nodes. By having lymphatic uptake as opposed to systemic absorption, the HA nanoconjugates provide for lower organ and systemic toxicity compared to current chemotherapy delivery technologies with naked drugs.

The molecular weight of the HA can be varied, but has a significant effect on uptake into the lymph system and thereby affects the lymphatic drug concentration. It has been found that hyaluronan has, superior performance at about 35 kDa, but can also be used at about 75 kDa, 150 kDa, or even 200 kDa for administration. Due to inflammatory responses, less than 10 kDa may not be feasible, and due to high viscosity more than 700 kDa may not be practical.

Accordingly, the molecular weight of HA can be optimized to about 10 to 200 kDa, more preferably about 20 kDa to about 150 kDa, still more preferably from about 25 kDa to about 100 kDa, and most preferably from about 30 kDa to about 75 kDa. These lower molecular weight HA polymers can be further refined depending on the drug being loaded and the accumulation characteristics of the nanoconjugate in the lymphatic system. For example, molecular weights of 30 kDA to 50 kDa can be advantageous as well as about 35 kDa polymers. These HA polymers are sufficiently soluble so as to be capable of transporting the drug conjugated thereto into the lymphatic system.

Also, the nanocarrier can be a dendrimer. The dendrimer generation can be selected to optimize the ratio of lymphatic to capillary uptake. Dendrimer nanoparticles have extremely well defined size and surface charge depending on the generation of material and the termini group chemistry. An example includes PAMAM dendrimers (polyamidoamine), phosphoester dendrimers, bis(3-hydroxypropyl) phosphonate dendrimers, Carboxy ester dendrimers, amino acid dendrimers, hyperbranched polymers (e.g. branched polyamino acids, branched polyesters, branched polyphosphoesters), polysaccharides (hyaluronan, dextran and its sulfonated derivatives, cellulose), and the like.

The nanoconjugate can be formulated for peritumor and subcutaneous injection for preferential translocation into the lymphatic system so systemic exposure is limited. The nanoconjugate can be from about 10 to about 30 nm to avoid capillary uptake with a neutral or negative charge to maximize rapid lymphatic uptake, preferentially about 15 to 25 μm, and most preferentially about 20 nm. There is an optimum size range for lymphatic uptake of subcutaneously injected particles: particles larger than 1.00 nm will remain largely confined to the site of injection, particles of about 10 to 80 nm are taken up by the lymphatics, and small particles and molecules (<20 kDa) will be absorbed by the blood capillary network into systemic circulation. Nanoconjugates larger than 100 nm or less than 5 nm are not very practical. Preferably, the nanoconjugates can be between about 10 and 80 nm (e.g., 10, 20, 30, 40, 50, 60, 70, or 80 nm), more preferably between about 15 and 50 nm, and most preferably between about 20 and 40 nm.

Previous reports have demonstrated the ability of HA to form stable conjugates with platinum drugs; however, the nanoconjugates of the present invention have different characteristics, such as molecular weight of HA, drug loading, and formulations configured for subcutaneous administrations. Never before have HA-drug conjugates been designed and formulated for subcutaneous administration for lymphatic deposition and retention as well as for suitable systemic concentrations. Furthermore, subcutaneous HA nanoconjugates have now been found to be drained to the axilla basin of rats after subcutaneous injection into the mammary fatpad. Thus, the compositions can be configured for direct injection into a tumor and/or subcutaneous injection for accumulation in the lymph system to treat metastasizes that may be found in the lymph system, such as in lymph nodes.

For example, subcutaneously injected cisplatin-HA nanoconjugates contained up to 0.25 w/w of cisplatin and released drug with a half-life of 10 hours in saline. Cisplatin-HA nanoconjugates had high anti-tumor activity in vitro similar to free cisplatin: cisplatin-HA IC₅₀ 7 μg/mL in MCF7 and MDA-MB-231 human breast cancer cells (free cisplatin IC₅₀ 7 μg/mL). Cisplatin-HA conjugates were well tolerated in rodents with no signs of injection site morbidity or major organ toxicity after 96 hours. The AUC of cisplatin in the axially lymph nodes after injection with cisplatin-HA increased 74% compared to normal cisplatin.

The systemic concentration of the chemotherapeutic drug delivered by the nanoconjugates can achieve a high enough level to be effective in treating any metastasized or systemic cancerous cells with a low enough level to be substantially non-toxic. Previously, the inclusion of naked platinum drugs in chemotherapeutic regimens has been associated with several toxicities including increased risk of leukopenia, nausea, hair loss, acute nephrotoxicity, chronic neurotoxicity, and anemia. As such, a loco-regional therapy approach for cancers confined to the breast and axilla may greatly improve the use of platinum drugs in breast cancer chemotherapy. For this purpose, hyaluronan may be an ideal carrier for localizing cisplatin to the lymph nodes.

In one embodiment, the nanocarrier is conjugated to the chemotherapeutic drug via a biodegradable linker. That is, the linker can be configured to degrade so as to release the chemotherapeutic proximal or within cancer cells. In the case of HA, the linker can be an acid-degradable linker. An acid-degradable linker can be utilized with HA because of the ability of HA to be internalized into a cell and translocated to a lysosome, which acidifies and degrades such a linker. Also, the hypoxic microenvironments around cancerous cells can degrade these linkers. This releases the drug directly into the cancerous cells that internalize the HA nanoconjugate.

Examples of acid-degradable linkers include hydrazone, esters, ketals, biodegradable polymer linkers, polylactide, polyglycolide; copolymers thereof, combinations thereof, and the like. In addition to acid degradable disulfides, 1,6 elimination linkers, phosphoester linkers, enzymatically cleavable linkers including but not limited to short peptide sequences recognized by enzymes found in tumors and surrounding tissues, lymphatics, and lymph nodes, which may be expressed at a higher level in these tissues than in most non-target tissues.

In one embodiment, the nanoconjugate is not pegylated. As such, the nanoconjugate can be substantially devoid of a PEG. Also, the nanoconjugate can be devoid of HPMA or polyglutames. The nanoconjugates can be formulated without being encapsulated.

The ability to subcutaneously administer the nanoconjugates and provide localized chemotherapy in the lymph system allows for the treatment of various cancers. More particularly, it allows for the treatment of early stage cancers that have begun to translocate through the lymph system. Thus, this administration route and accumulation in the lymph system allows the nanoconjugates to provide localized therapy to a variety of cancers, such as breast cancer, colon cancer, lung cancer, non-small cell lung, melanoma, head and neck cancers (e.g., head and neck squamous cell carcinoma), ovarian cancer, and lymphoma as well as others.

Direct injection into rat breast tissue of cisplatin with a silver-activated nanoconjugate of cisplatin was studied even though local injection of cisplatin is not feasible due to tissue damage. Additionally, tumor studies showed the silver activated nanoconjugate to cause premature animal death. As such, the nanoconjugate of the present invention was developed and it does not require the use of silver (i.e., the nanocarrier is not silver activated), and thereby the nanoconjugate of the present invention does not have the toxic side effects associated with silver. The localized chemotherapy with silver-free nanoconjugate chemotherapeutics (e.g., HA-cisplatin nanoconjugates) after subcutaneous administration was compared to standard intravenous administered cisplatin with respect to the major organ pathologies in response to the different treatments. Subcutaneous administration of the nanoconjugates provided localized nanoconjugate chemotherapy with significantly increased lymphatic tissue concentrations over systemic therapy and reduced organ toxicities including nephrotoxicity in rats.

Previously, hyaluronan was activated with silver nitrate prior to conjugation with cisplatin, as this has been reported to improve conjugation efficiency. However, it has now been found that it is extremely difficult to remove all traces of silver from the resulting conjugates, even after multiple rounds of extended dialysis against water. The presence of silver in the treatments resulted in a significant number of animals succumbing to silver-induced toxicity as determined by pathological examination. This is unacceptable in human treatment, and led to an alteration of the formulation schema to eliminate silver from the conjugation procedure. The coupling reaction was then re-engineered without silver activation in order to obtain the highest conjugation efficiency, which ultimately did not impair formation of the HA-platinum conjugates. This is a significant advancement in terms of clinical development as cisplatin and HA are both approved by the FDA for use in humans and no additional substances are required for formation of the complex. The resulting nanoconjugates still had excellent antiproliferation activity against multiple breast cancer lines in vitro, indicating the change in formulation method does not affect cell-based drug efficacy.

The pharmacokinetics and tissue distribution for the nanoconjugate indicated lymphatic delivery of the HA-cisplatin nanoconjugate improved drug levels in the local lymph basin compared to intravenous cisplatin dosing. At an equivalent dose of platinum, the HA-cisplatin carrier greatly increased lymph node basin concentrations, suggesting the carrier is able to deliver platinum to the lymph nodes through the lymphatics much more effectively than intravenous drug administration routes. In addition, the HA-cisplatin nanoconjugate appeared to maintain its stability in vivo long enough to traffic or localize into the lymphatics before releasing its conjugated drug.

The nanoconjugate preferential accumulation in the lymphatic system reduces systemic tissue exposure to platinum compared to intravenously delivered cisplatin, but the HA-cisplatin nanoconjugate due to its sustained release properties (e.g., selective translocation and selective degradation of the linker) actually increased platinum AUC an average of 200% in most tissues compared to intravenous cisplatin. This, is likely clue to the accumulation of platinum (front the HA-cisplatin delivered subcutaneous) over time from a more sustained release profile compared to rapid decay and elimination via an intravenous bolus infusion. This increased tissue level also carries two advantages: (1) a lower dose of platinum being required to achieve the same tissue effect such that the HA-cisplatin dose may be reduced 50% and still maintain equivalent tissue levels; and (2) maintaining therapeutic systemic levels of drug is important for utilizing this drug as an adjuvant therapy since it is well known that most patients with breast cancers which have metastasized to the loco-regional lymph nodes have likely micro if not macrometastasis systemically. Therefore, this treatment can be use in place of daily systemic intravenous therapy by now utilizing a less invasive and less-frequent dosing schedule, e.g., weekly subcutaneous dosing as compared to current therapy which is daily intravenous infusion, while simultaneously providing a local “boost” of drug delivery to the loco-regional tumor basin and lymphatics. The larger AUC of HA-cisplatin can also increase rates of tumor apoptosis since prolonged subtoxic levels of cisplatin can substantial improve tumor cell apoptosis compared to a single high dose.

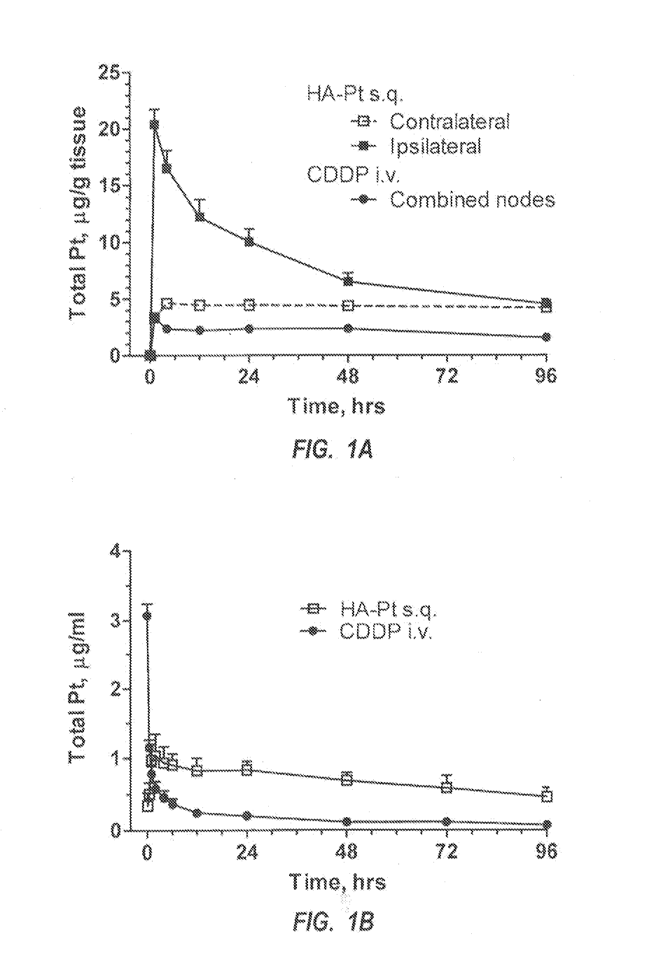

Since the more severe side effects of cisplatin are likely due to the high peak plasma levels (Cₘₐₓ) experienced immediately after intravenous administration, recent applications included metronomic dosing regimens have been shown to decrease toxicity although they increase inconvenience and costs to patients. As such, locally administered nanoconjugates prevent high peak levels both due to slow release of drug from the carriers, with a half-life in saline of around 10 hours compared to under an hour for the standard intravenous cisplatin formulation, and after its release from the carrier, the cisplatin takes time to diffuse from the tissues or lymph into the systemic circulation. Subcutaneous HA-cisplatin has a much lower peak plasma concentration compared to intravenous cisplatin (FIGS. 1A-1B), although the overall plasma platinum AUC is much greater than with intravenous cisplatin. After subcutaneous HA-cisplatin injection, there was a 2 hour delay before plasma platinum peaked and plateaued-off, then remaining at a constant level. This release profile is consistent with a delayed and sustained release from the lymphoid tissues.

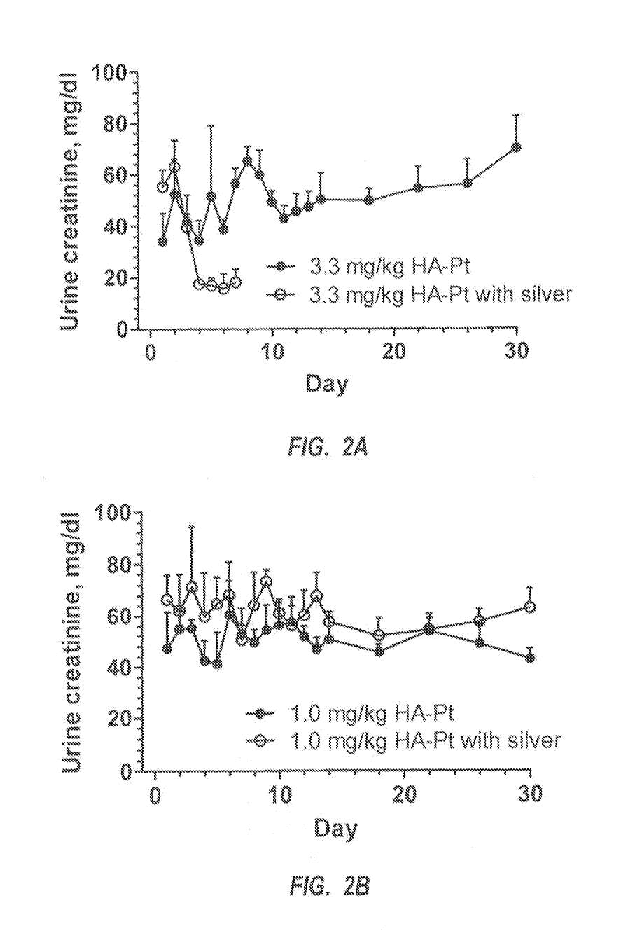

FIGS. 2A-2B show that creatinine levels did not indicate significant differences in renal toxicity between intravenous cisplatin and localized nanoconjugates despite the much higher AUC of nanoconjugate platinum. The high dose silver formulation caused decreased urine creatinine levels and animal death within the first week, which is unfavorable. The severe in vivo toxicity of silver formulations indicates that the slightly greater in vitro anti-proliferative activity of silver formulations (Table 1) may be due to non-specific silver toxicity. Although no damage was detected, creatinine testing may not be sensitive enough to detect minor renal damage after a single moderate dose, so pathological examinations were conducted for more direct evidence of platinum toxicity. Thus, in one embodiment, a nanoconjugate formulation is substantially devoid of silver.

Pathological examination 30-days following a single dose injection of cisplatin revealed there were significant renal and hepatic differences between the subcutaneous HA-cisplatin and intravenous cisplatin formulations. Although the renal platinum AUC of HA-cisplatin was twice that of cisplatin, the lower incidence and less severe nature of renal cell necrosis in the HA-cisplatin treated animals indicated improved tolerability over intravenous cisplatin. The greater platinum AUC of HA-cisplatin in the kidneys would seem to contradict the lower toxicity observed, but may be due to its lower peak serum drug levels (Cₘₐₓ) filtered by the kidney compared to intravenous cisplatin.

Liver pathology indicated decreased toxicity of the HA-cisplatin compared to cisplatin, and none of the animals had normal pathology in the 3.3 mg/kg cisplatin arm. Cisplatin-induced hepatotoxicity is known to occur due to the production of reactive oxygen species. Hyaluronan is metabolized in the liver and glycosaminoglycans are known antioxidants with hepato-protective effects, so the HA nanoconjugate may protect against platinum hepatotoxicity.

Accordingly, an embodiment includes a nanoconjugate of HA and cisplatin that concentrates cisplatin in the breast lymphatics after subcutaneous injection into the mammary fatpad. These nanoconjugates have sustained release characteristics resulting in a higher lymphatic AUC and lower plasma Cₘₐₓ compared to standard intravenous cisplatin. The nanoconjugates do not cause substantial organ toxicities, such as renal, hepatic, neuro, or nephrotoxicity; and on pathological examination appear to have lower organ toxicity compared to the standard intravenous cisplatin. The nanoconjugates do not cause injection site or lymph node toxicities. The preferential intralymphatic translocation and accumulation of the nanoconjugates provides advantages for use in combination regimens for breast cancer with other chemotherapeutics.

In accordance with the present invention, the nanoconjugate can deliver cisplatin effectively to be use alone or as part of a combination therapy with significantly less toxicity. The intralymphatic delivery model using nanoconjugates not only increases drug concentrations in loco-regional nodal tissue significantly above the standard cisplatin formulation (74% greater AUC), but it also exhibits sustained release kinetics, allowing lower Cₘₐₓ levels which lower organ toxicity over time. The only tissue level that was significantly different was the axillary lymph nodes ipsilateral to the drug injection. This translated into almost double the concentration of cisplatin penetrating the loco-regional nodes using nanoconjugates injected directly into the breast subcutaneously. Therefore, nanoconjugates that are preferentially translocated to the lymphatics significantly boosts the concentration of drug to treat and/or inhibit loco-regional tumor cell development in the lymphatics.

Also, the nanoconjugates can be successful in treating and/or inhibiting the spread of cancer because of the intralymphatic delivery of chemotherapeutics using hyaluronan as a targeted nanocarrier to the axilla. The preferential intralymphatic delivery can preferentially treat at-risk regional lymph nodes and avoid systemic toxicities associated with intravenous or oral drug administration. The preferential intralymphatic delivery reduces the systemic concentration but maintains a suitable level for also treating and/or inhibiting the spread of cancerous cells that are disseminated into the systemic circulation. As such; the nanoconjugates can provide a therapy for patients with sentinel nodes containing nanometastases which would not be offered lymph node dissections routinely. The nanoconjugate provides adequate systemic drug levels in a more sustained-release manner than standard therapy, but it also provides a much-needed boost to the loco-regional nodal tissue, which is at risk for harboring tumor cells not removed by nodal dissection. Additionally, the nanoconjugate can be used as a neoadjuvant for locally advanced breast cancers, and can treat or inhibit regression.

In one embodiment, a pharmaceutical formulation having the nanoconjugate is not formulated for the following delivery routes: oral, systemic, transdermal, intranasal, suppository, intravenous, or intraluminal administration.

The pharmaceutical can be configured for percutaneous, intradermal, mucosal or submucosal, subcutaneous, interstitial, intrafat, peritumoral, intramuscular injection mucosa, peritumorally, inhalation, and instillation.

The nanoconjugate of the present invention preferentially translocating to the lymphatic system after subcutaneous or interstitial administration is surprising and unexpected. In part, this is due to the preferential translocation and accumulation in the lymphatic system after subcutaneous or interstitial injection, such as into the breast tissue. The nanoconjugate of HA or dendrimer with a chemotherapeutic such as cisplatin additionally provides a therapeutic systemic dose with AUC similar or sometimes higher than standard intravenous agents (e.g., cisplatin and doxorubicin) but lower Cₘₐₓ concentrations. This combination of findings allows these drugs to be used as superior adjuvant therapies for patients with loco-regional disease, with the additional benefit of being able to treat their systemic disease and with less toxicity since the Cₘₐₓ is lower and this is associated with cisplatin and doxorubicin toxicity. It is also surprising that the HA nanoconjugates were substantially less toxic than standard cisplatin or doxorubicin with regard to local injection site; kidney, and ototoxicity evaluation by OAEs and by renal pathologic analysis. The lower toxicity allows for direct injection into, adjacent, or proximally into tissue or interstitial space without damaging the healthy tissue. Thus, the nanoconjugates can provide therapy to the primary tumor, the intralymphatic cancerous cells, and systemic cancerous cells.

In summary, the nanocarrier delivery system is superior to standard drug formulations in (1) its efficacy and ability to treat cancers in animals, (2) its lower toxicity profile, and (3) its longer dosing interval (weekly or biweekly versus daily with intravenous agents). The subcutaneous injection offers patients a less invasive treatment option than being attached to intravenous infusion pumps which carry the risk of drug extravasation.

The nanoconjugate can be included in a pharmaceutical composition with an acceptable carrier that formulates the nanoconjugate for suitable administration, such as subcutaneous. Suitable preparations for subcutaneous administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

According to the methods of the present invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced, using any mode of administration that is medically acceptable, meaning any mode that produces effective levels of the active composition without causing clinically unacceptable adverse effects.

For injection, the nanoconjugates can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. Preferably, the nanoconjugates can be formulated in aqueous solutions, preferably in physiologically compatible buffers such as Hanks's solution, Ringer's solution, or physiological saline buffer.

The nanoconjugates can be formulated for subcutaneous administration by injection, e.g., by bolus injection or continuous infusion. Formulations for injection may be presented in unit dosage form, e.g., in ampules or in multidose containers, with an added preservative. The compositions may take such forms as suspensions, solutions, or emulsions in oily or aqueous vehicles, and may contain formulator agents such as suspending, stabilizing, and/or dispersing agents.

Sterile injectable forms of the compositions of this invention may be aqueous or a substantially aliphatic suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The involvement of the lymphatic system in breast cancer metastasis is well established, yet there are no effective non-surgical treatments to overcome lymph node metastases and disease progression. The greatest challenge with chemotherapy of lymphatic metastases is maximizing the amount of agent which actually is retained in the lymph nodes while avoiding systemic absorption and toxicity.

The lymphatically-localized chemotherapy provided by the present invention is an innovative leap in breast cancer therapy. The HA or dendrimer nanocarriers for chemotherapeutic drugs can treat locally advanced breast cancer utilizing both a targeted and lymphatic delivery approach. The use of lymphatic targeted nanocarriers for intralymphatic drug delivery in breast cancer is highly innovative and has never been performed to date. By increasing drug loco-regional AUC over that achievable by standard chemotherapy drugs this technology provides significant neoadjuvant therapy in locally advanced breast cancer. Additionally having a targeted approach with better retention and sustained release of drug from the lymphatics should decrease systemic toxicity of the drugs leading to a combination of better tumor efficacy and lower toxicity.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited and/or shown herein are incorporated herein by specific reference.

### EXPERIMENTAL

Hyaluronan from microbial fermentation was purchased from Lifecore Biomedical (Chaska, Minn.) as sodium hyaluronate and used without further purification. Heparin solution was purchased from Abraxis Pharmaceutical Products (Schaumburg, Ill.). All other reagents were purchased from Fisher Scientific (Pittsburgh, Pa.) or Sigma Aldrich (St. Louis, Mo.) and were of ACS grade or better. Milli-Q water was used in all experiments. The MDA-MB-468LN cell line was kindly provided by Ann Chambers (London Health Sciences Center, London, Ontario), while MCF-7 and MDA-MB-231 cells were obtained from the American Tissue Culture Collection (ATCC, Manassas, Va.). Animal procedures were approved by the University of Kansas Institutional Animal Care and Use Committee. Sprague-Dawley rats were purchased from Charles River Laboratories (Wilmington, Mass.).

1. Synthesis of HA-Cisplatin Conjugates

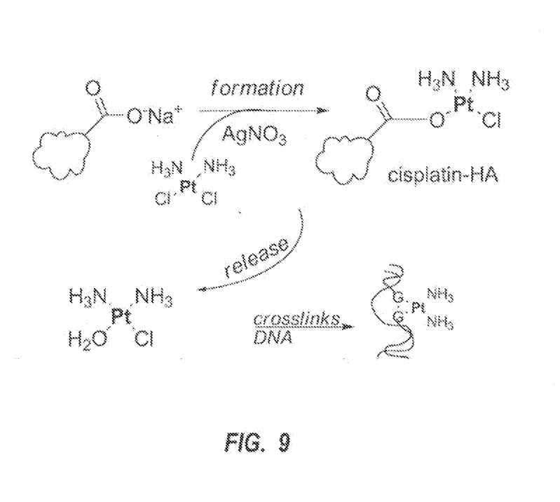

Cisplatin was conjugated to HA (35.000 g/mol) based on previously reported procedures (Cai, et al., Intralymphatic chemotherapy using a hyaluronan-cisplatin Conjugate, J. Surg. Res. 147 247-252 (2008); Jeong, et al., Cisplatin-incorporated hyaluronic acid nanoparlicles based on ion-complex formation, J Pharm Sci. (Epub. 2008), with and without the addition of silver nitrate as an activating agent. Typically, HA (100 mg) and cisplatin (45 mg) were dissolved in H₂O (20 mL) and stirred in the dark for four days under argon at ambient temperature (ca. 25° C.). The reaction mixture was filtered (0.2 μm nylon membrane) and dialyzed against H₂O (10,000 MWCO; Pierce, Rockford, Ill.) for 48 hours at 4° C. protected from light. Following dialysis, the crude product was concentrated and stored at 4° C. The degree of cisplatin substitution was determined by atomic absorption spectroscopy (“AAS”) (Varian SpectrAA GTA-110 with graphite furnace). The furnace program, was as follows: ramp 25 to 80° C., hold 2 seconds, ramp to 120° C., hold 10 seconds, ramp to 1000° C., hold 5 seconds, ramp to 2700° C., hold 2 seconds, cool to 25° C. over 20 seconds. The graphite partition tube was cleaned every 40 samples by baking at 2800° C. for 7 seconds. Argon was used as the injection and carrier gas. The resulting conjugate is referred to as HA-cisplatin, cisplatip-HA, IA-CDDP, HA-Pt, although the conjugate is [PtCl(H₂O)(NH₃)₂]OOCO-HA (FIG. 9).

The structure of cisplatin lends itself to complex formation with polycarboxylic polymers, since one or more of the chlorides can be displaced allowing formation of a labile ester linkage with the polymer. Cisplatin was highly conjugated to HA with typical conjugations of 0.20 w/w platinum/complex (approximately 65% cisplatin conjugation efficiency). In previous studies, cisplatin conjugates were synthesized by first activating HA with AgNO₃; however, it has now been found that eliminating this step does not significantly reduce conjugation and it reduces potential silver toxicity. The AAS produced a linear curve in the range of 10 to 450 ng/mL (R²=0.999) with a limit of detection of 5 ng/ml. Concentrated samples were diluted with water into the linear analytical range prior to analysis.

Fluorescent conjugates of HA were formed by condensation of Texas Red hydrazide to HA. HA (35 000 MW, 100 mg) in 10 mL of 30% H₂O:EtOH was activated with 2-chloro-1-methylpyridinim iodide (33 mg) and triethylamine (35 μL). After the addition of Texas Red hydrazide (AnaSpec Inc., San Jose, Calif.) (2 mg in 0.4 mL of DMSO), the mixture was refluxed for 24 hours. Workup proceeded by dialysis against H₂O for 48 hours at ambient temperature, followed by lyophilization. Conjugation efficiency was determined using a molar extinction coefficient of 81 800 M⁻¹cm⁻¹ at λ 588 nm.

Cisplatin was highly conjugated to HA, with typical Conjugations of 0.25 w/w cisplatin/complex using a starting ratio of 0.5 w/w cisplatin/HA. Up to 0.75 w/w cisplatin/complex was attempted with decreasing efficiency (Table 4).

2. In Vivo Cell Toxicity

The lymphatically metastatic breast cancer cell line MDA-MB-468LN was maintained in modified Eagle's medium alpha supplemented with 10% fetal bovine plasma, 1% L-glutamine, and 0.4 mg/mL G418 (geneticin). Additional breast cancer cell lines MDA-MB-231 and MCF-7 were maintained according to protocols provided by the ATCC. Preceding proliferation studies, cells were trypsinized and seeded into 96-well plates (5,000 cells/well). After 24 hours, cisplatin, HA-cisplatin (with or without silver activation), or HA was added (n=12; 7 concentrations), and 72 hours post-addition, resazurin blue in 10 μl of phosphate-buffered saline was added to each well (final concentration of 5 mM). After 4 hours, well fluorescence was measured (λₑₓ 560 nm, λₑₘ 590 nm) using a fluorophotometer (SpectraMax Gemini; Molecular Devices, Sunnyvale, Calif.). IC₅₀ was determined as the midpoint between saline (positive) and cell-free (negative) controls for each plate.

Cell toxicity was determined as the reduction in cell proliferation over 72 hours. HA-cisplatin conjugates with and without silver had similar cytotoxicity to free drug in cell culture (Table 1). No appreciable difference in toxicity was detected between cisplatin and HA-cisplatin using three different human breast cancer cell lines (Table 1). HA showed no toxicity at 10 mg/ml, the upper limit of testing in all cell lines compared with saline controls (data not shown).

3. Pharmacokinetics and Tissue Distribution

Sprague-Dawley rats (female, 200-250 g) were cannulated in the left jugular vein under isoflurane and allowed to recover overnight. Animals were then injected intravenous with cisplatin (1.0 or 3.3 mg/kg; n=5) or subcutaneous with HA-cisplatin (1.0 or 3.3 mg/kg equivalent cisplatin; n=5) under isoflurane anesthesia. Subcutaneous injections were given in the uppermost right mammary fatpad of the animal. Whole blood was withdrawn (100 μl) from the cannula at 0, 5 minutes, 0.5, 1, 2, 4, 6, 12, 24, 48, and 96 hours after closing and placed into 2-ml centrifuge tubes pretreated with heparin. The cannula was washed before and after withdrawal with saline and then heparin locked. The whole blood was centrifuged at 17,000×g for 5 minutes, and the plasma was frozen at −80° C. until analysis. Animals were euthanized 96 hours after treatment. The right ipsilateral axilla nodes (treated side), left contralateral axilla nodes (control side), and major organs (liver, kidneys, heart, spleen, lungs, brain, muscle, bladder) were excised; washed with 0.9% saline; and stored at −80° C. until analysis. Tissue samples were prepared using a procedure reported previously (Cai, et al., Intralymphatic chemotherapy using a hyaluronan-cisplatin conjugate, Surg. Res. 147:247-252 (2008)). Typically, 50 mg of tissue sample was digested using 1.5 ml of 6.7% nitric acid for 2 hours at 80° C. After digestion, samples were homogenized (Tissue Tearor; BioSpec Products Inc., Bartlesville, Okla.) and centrifuged. The supernatant and plasma samples were analyzed by AAS as described in the Synthesis section. The pharmacokinetics of subcutaneous HA-cisplatin were compared to intravenous cisplatin in Sprague-Dawley rats. HA-cisplatin accumulated more preferentially in the draining ipsilateral axillary lymph nodes than did the intravenous cisplatin control (FIG. 1A); preferential accumulation was still evident at 48 hours post-injection even though the in vitro disassociation half-life of cisplatin from HA is 10 hours. The ipsilateral axillary node AUC_{0-96 hrs} of HA-cisplatin when injected locally was 3.8-fold greater than intravenous cisplatin (p<0.001), and the peak node concentration (Cₘₐₓ) of HA-cisplatin was 6.2-fold greater than intravenous cisplatin.

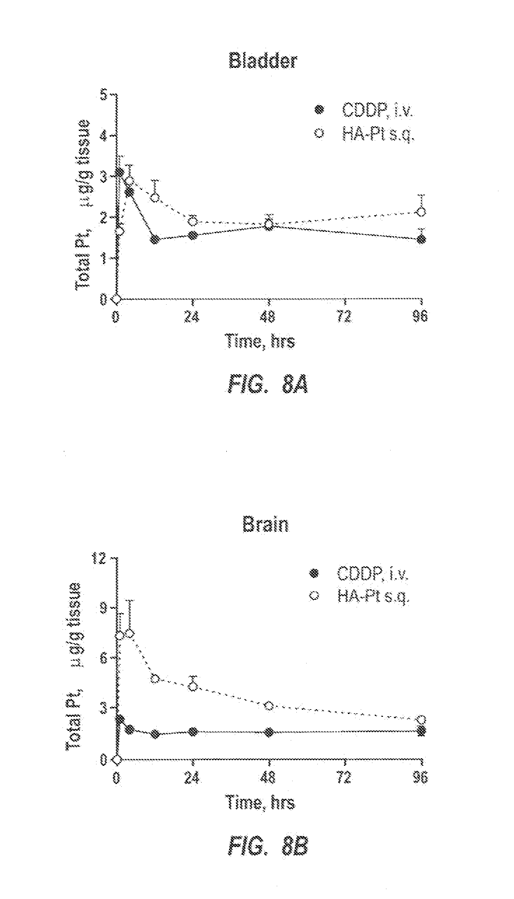

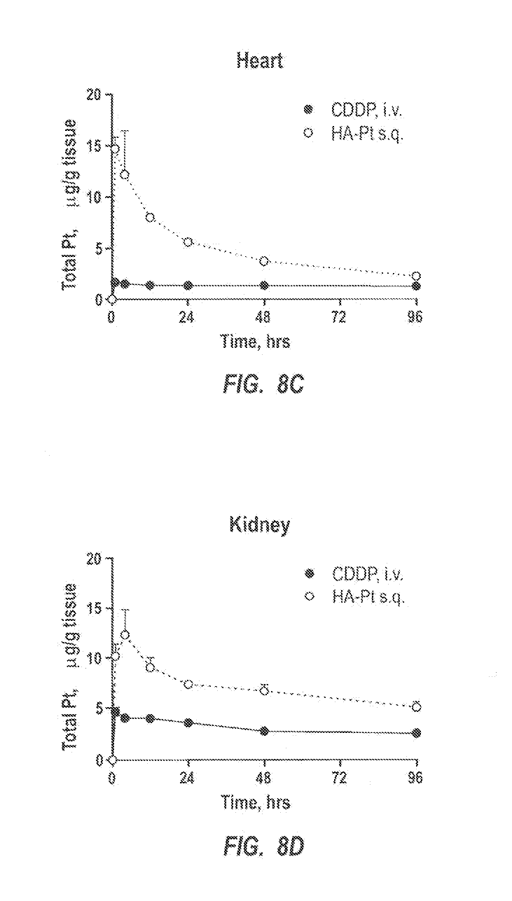

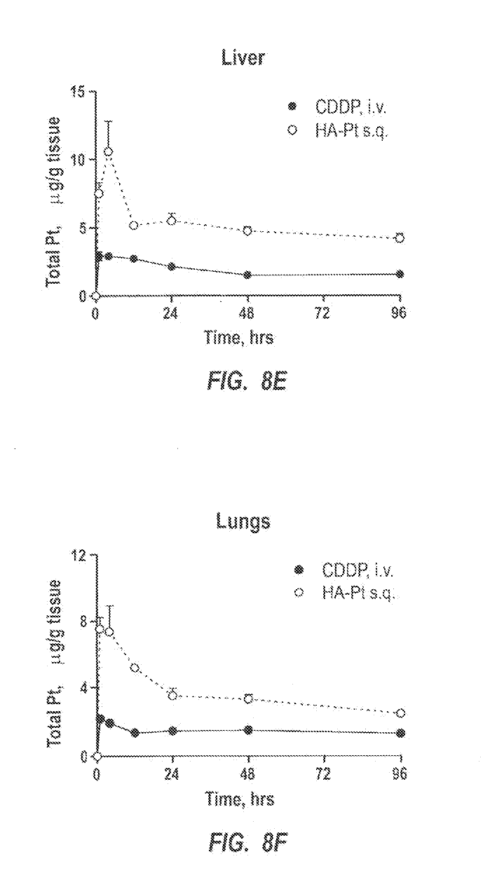

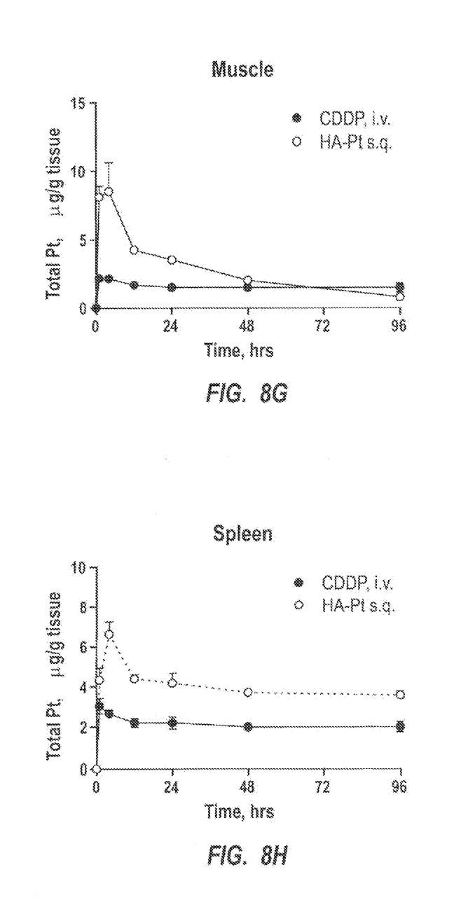

The most significant, dose-limiting toxicities of cisplatin therapy are nephrotoxicity followed by neurotoxicity, both of which are strongly influenced by peak plasma concentration. The peak plasma concentration intravenous cisplatin was 3.1-fold greater than subcutaneous HA-cisplatin. The release of cisplatin into the systemic circulation was slow, and the resulting plasma AUC of HA-cisplatin was 3.9-fold greater than intravenous therapy with cisplatin, which is consistent with longer lymphatic retention of the nanocarrier HA-cisplatin (FIG. 1B, Table 2). Concentration graphs for all tissues are included in supplement (FIG. 8A-8H).

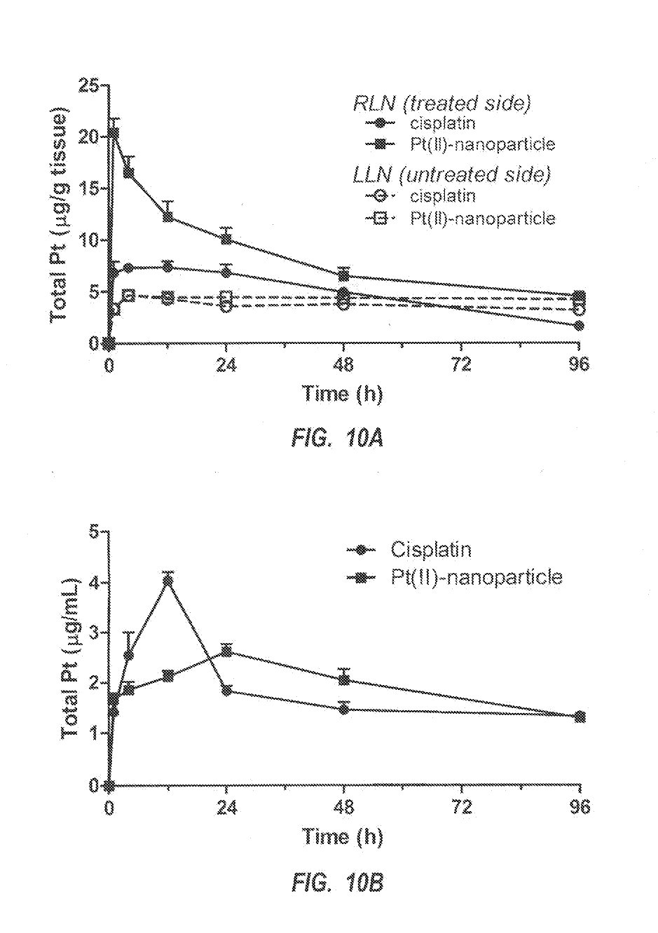

Additionally, Sprague-Dawley rats (200-250 g females, Charles River) were placed under isoflurane anesthesia and injected subcutaneously (100 μL) into the right mammary fat pad with cisplatin-HA or cisplatin in 0.9% saline (3.5 mg/kg equivalent cisplatin) (n=5). Animals were allowed to recover with access to food and water. After 1, 4, 12, 24, 48, and 96 hours post-injection, animals were euthanized by isoflurane overdose. Organs and tissues were washed with 0.9% saline and frozen (−80° C.) until analysis. Plasma was separated by centrifugation from whole blood and frozen (−80° C.). Conjugation of cisplatin to HA impacted the local concentration of cisplatin in draining lymph nodes with a minor effect on systemic concentrations (FIGS. 10A-10B, Table 5). Over the experimental timeframe of 96 hours, the area-under-the-curve (“AUC”) of cisplatin-HA conjugates in the right lymph node (“RLN”), which drains the injection site, was 74% greater than cisplatin in saline (p=0.0001), and the RLN had increased tissue concentrations over the examined period (FIG. 10A). The AUC of cisplatin-HA in the non-draining left lymph node (“LLN”) was not significantly different from cisplatin in saline (p=0.12).

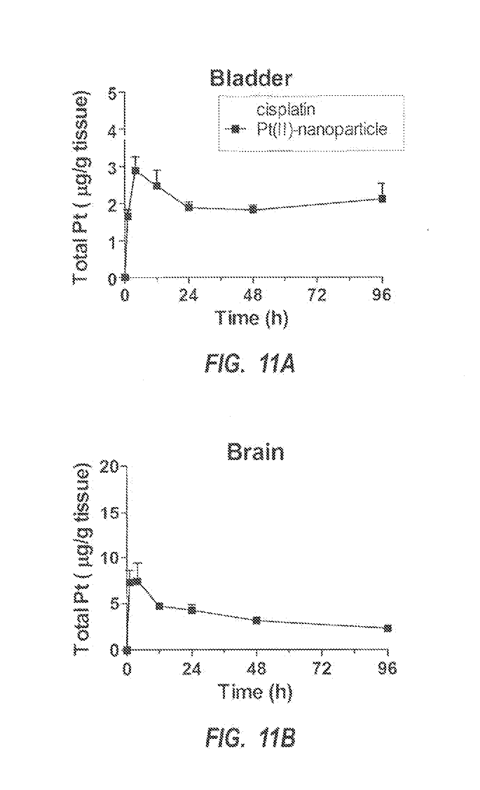

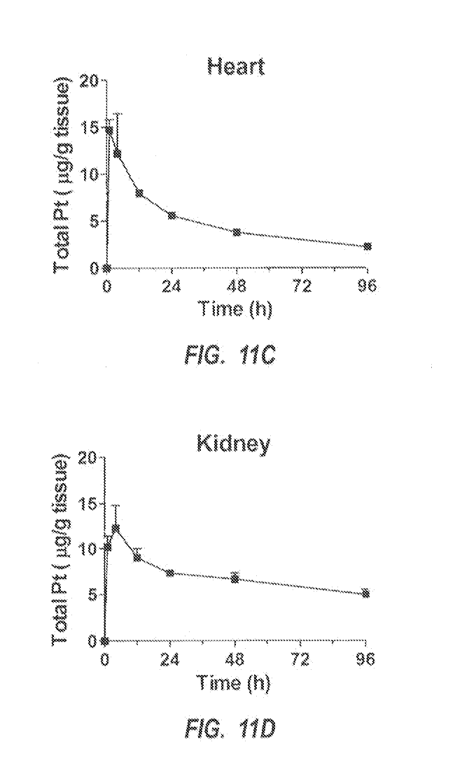

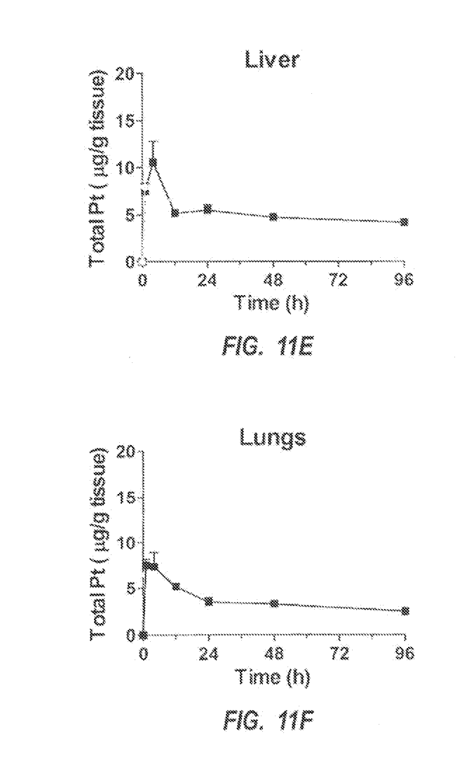

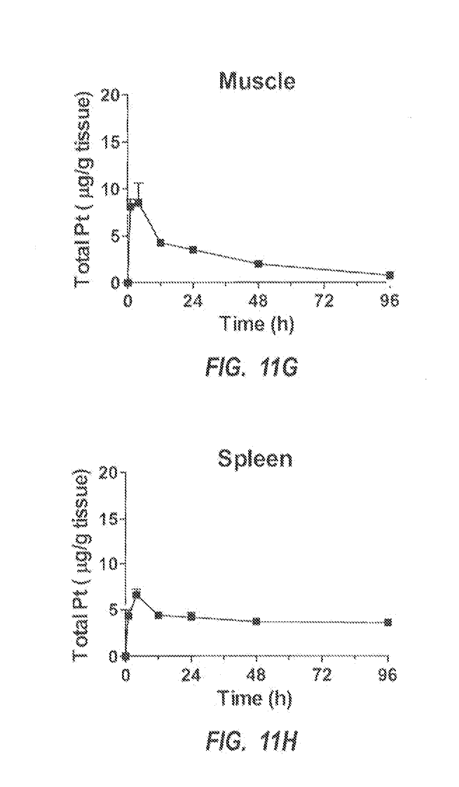

A burst release of free cisplatin appeared in the plasma concentration profile (FIG. 10B), whereas cisplatin-HA demonstrated a longer, sustained release into the plasma. This is significant because close-limiting toxicities of cisplatin therapy are strongly influenced by peak plasma concentration. There was not a significant difference in the plasma AUC between cisplatin-HA and cisplatin (p=0.13). Thus, localized therapy with cisplatin-HA may generate sufficient serum concentrations to treat distant metastases, while providing a boost therapy for the breast lymphatics. The distribution of cisplatin to other organs was not significantly different over the study period (FIG. 11, Table 5).

4. Long-Term Toxicology

Sprague-Dawley rats (35 females) were randomly divided into 7 study groups of 5 animals each: 1.0 mg/kg subcutaneous HA-cisplatin (with and without silver; platinum equivalent to 1.0 mg/kg cisplatin), 3.3 mg/kg subcutaneous HA-cisplatin (with and without silver), intravenous cisplatin at 1.0 and 3.3 mg/kg, and subcutaneous HA (control; HA equivalent to 3.3 mg/kg HA-Pt). Each animal was administered a single bolus dose at the beginning of the 30-day study period. Urine samples were collected every day during the first two weeks of the study and every four days during third and fourth week of the studies (except for the 3.3 mg/kg HA-cisplatin with silver group). In order to reduce the stress to animals, subjects were housed in metabolic cages for 12 hours to collect approximately 5 ml of urine and then returned to cages with bedding until the next collection period. Urine samples were centrifuged at 17,000×g for 5 minutes and stored in −80° C. freezer until creatinine analysis.

Urine creatinine was analyzed using the QuantiChrom™ Creatinine Assay Kit according to the manufacturer's instructions (BioAssay Systems, Hayward, Calif.). Creatinine concentration of the sample was calculated as (OD_{SAMPLE 5}−OD_{SAMPLE 1})/(OD_{STD 5}−OD_{STD 1})×[STD] (mg/dL). OD_{SAMPLE5}, OD_{SAMPLE1}, OD_{STD5}, and OD_{STD1} are OD_{510 nm} values of sample and standard at 5 minutes and 1 minute, respectively.

The animals were euthanized at the end of the study (30 days) and the liver, bilateral kidneys, spleen, lungs, heart, right (ipsilateral) and left (contralateral) axillary nodes, and brain were excised intact and stored in 80% alcoholic formalin solution overnight for fixation before slide mounting. Mounting using hematoxylin & eosin (“H&E”) staining were conducted by Veterinary Lab Resources (Kansas City, Kans.). The pathological examination was performed by a blinded board-certified veterinarian pathologist (University of Kansas Medical Center, Kansas City, Kans.).

Urine creatinine levels are an indirect indicator of renal function and renal toxicity, with a decrease in creatinine excretion corresponding to decreased renal function and possible renal toxicity or damage. Significant renal toxicity was observed in animals given the high dose silver regimen (3.3 mg/kg), with a 30% decrease in creatinine excretion at 3 days and 70% decrease at 4 days. All animals in this group died within 1 week of treatment due to drug-related cachexia. In contrast, the silver-free high dose HA-cisplatin did not demonstrate significant toxicity, and creatinine levels remained near pre-dosing levels throughout the study's duration. Similar renal function was observed in both groups when animals were administered low doses (1.0 mg/kg) of either HA-cisplatin with silver or HA-cisplatin without silver treatment (p>0.05, day 1 to 30) (FIGS. 2A-2B).

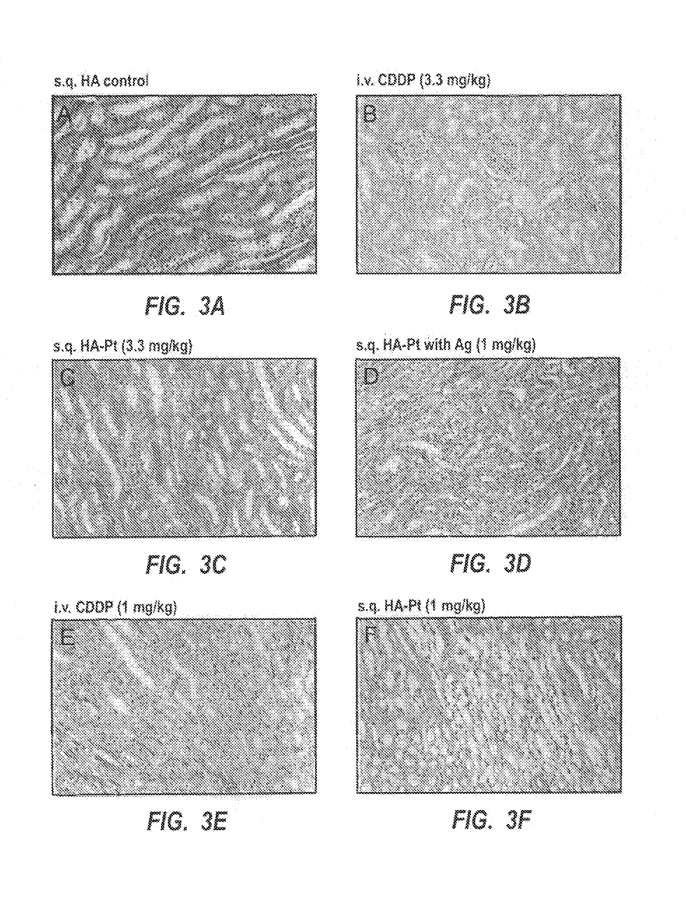

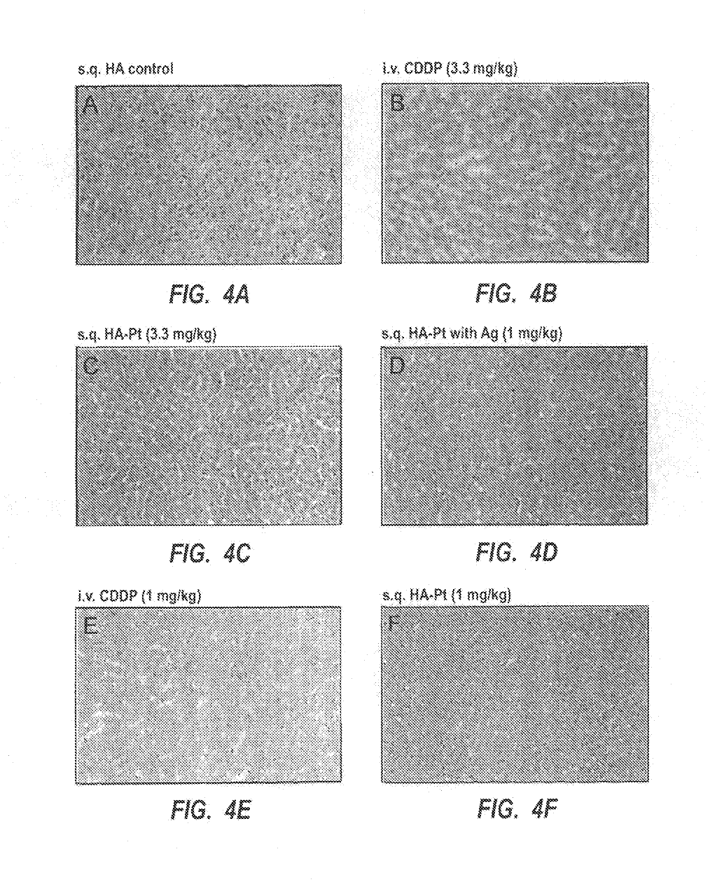

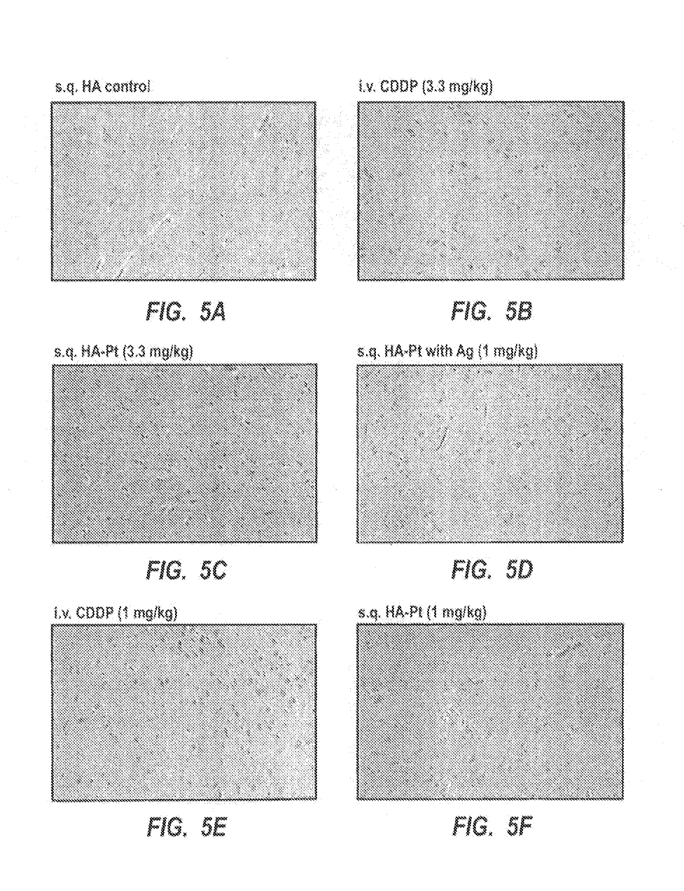

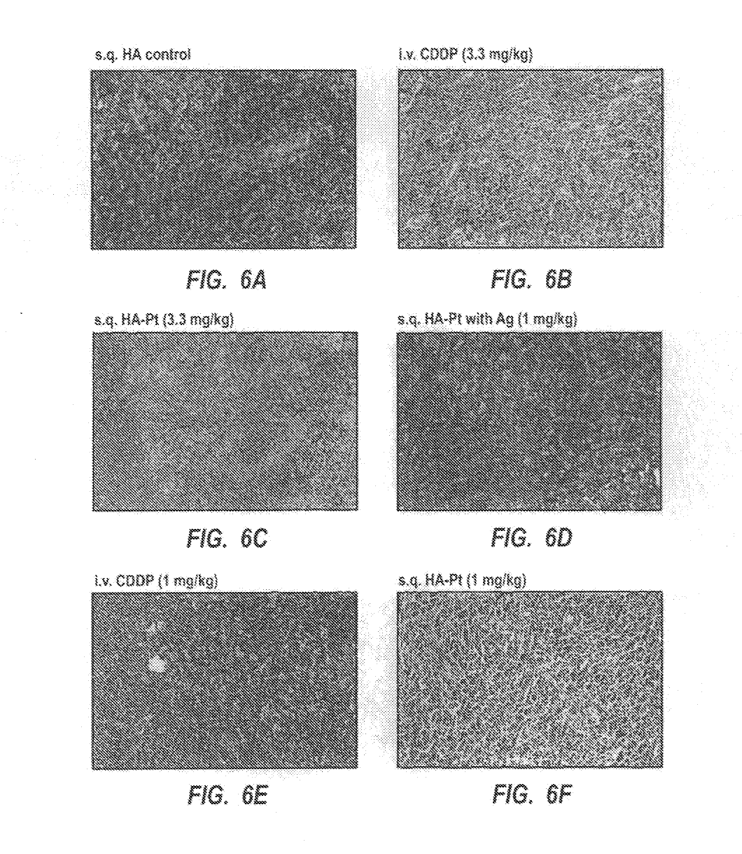

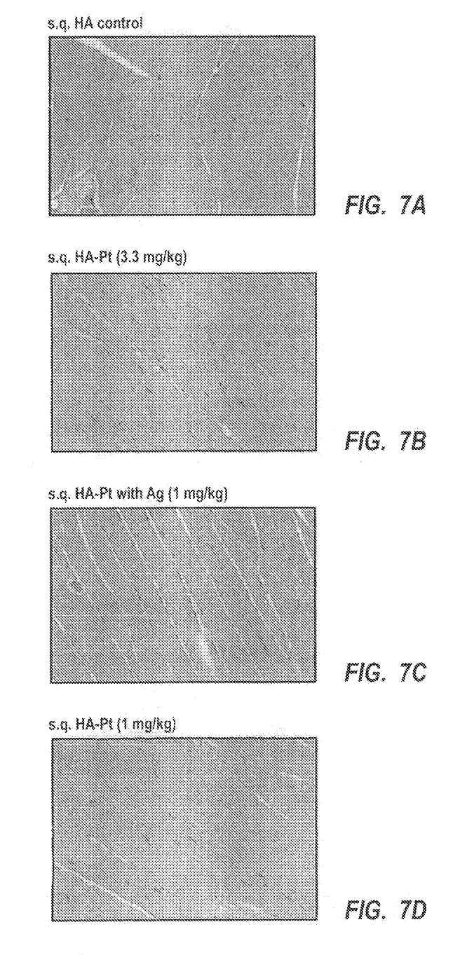

At the conclusion of the 30 day toxicity study, animals were euthanized and a full pathological examination performed. Brain tissue and underlying tissue of the injection site were normal with no microscopic changes for all study groups. Very mild changes in lymph nodes were detected for high dose intravenous cisplatin and subcutaneous HA-cisplatin formulated without silver. Lymphoid tissue from low dose subcutaneous HA-cisplatin with silver had normal appearance indicating by well-populated small lymphocytes showing little or no follicular architecture (FIGS. 6A-6F). Very mild changes were observed in the livers for animals receiving both low dose cisplatin intravenous and low dose HA-cisplatin subcutaneous indicated by the presence of mild inflammation in the sinusoids (FIGS. 4A-4F). Mild degeneration with some sinusoidal necroses were observed for animals receiving high dose intravenous cisplatin and high dose subcutaneous HA-cisplatin treatment; necroses were more severe in the intravenous cisplatin group. In addition, 60% of animals receiving low dose intravenous cisplatin were observed with mild renal necrosis including hemorrhage into the renal tubules along with tubular, edema (FIGS. 3A-3F). In contrast, none of the animals receiving low dose subcutaneous HA-cisplatin had renal necrosis. Similarly, 4 of 5 (80%) animals receiving high dose intravenous cisplatin compared to 1 of 5 (20%) animals receiving high dose subcutaneous HA-cisplatin were diagnosed with mild renal necrosis (Table 3). Overall, the pathology studies demonstrated that the silver-free HA-cisplatin conjugates demonstrated lower incidence of both renal and hepatic toxicity compared to the conventional intravenous cisplatin treatment at all dose ranges. Additionally no neurotoxicity in the brain or local injection site toxicity in the underlying muscle tissue, was observed in the treated animals (FIGS. 5A-5F and 7A-7D).

5. In Vitro Drug Release

In vitro release rate of cisplatin from cisplatin-HA was determined in phosphate buffer with and without saline. Cisplatin-HA was added to 3,500 MWCO dialysis bag (Pierce) and placed in a phosphate-buffered water bath (pH 7.4, 37° C.) or physiological saline (140 mM). Samples were taken from the dialysis bags at predetermined time points and remaining cisplatin concentration determined by AAS. The release rate of cisplatin from complexes was determined in both phosphate buffered saline and water. The in saline was expected to more rapidly displace cisplatin, increasing the release rate. The release of drug showed near first order release kinetics with a release half-life of 42 hours in water and 10 hours in physiological saline. The AAS produced a linear concentration curve from 10 to 450 ng/mL (R²=0.9998), with a limit of detection of 5 ng/mL, and a limit of quantification of 10 ng/mL (5% standard deviation). Cisplatin recovery from cisplatin-HA spiked tissues was: plasma, 82±4% (±STD); lymph nodes, 92±2%; bladder, 88±1%; brain, 94±0.3%; heart, 97±1%; kidneys, 98±1%; liver, 100±1%; lung, 94±1%; muscle, 95±1%; spleen, 97±1%. Cisplatin recovery from cisplatin-spiked tissues was: plasma, 80±3%; lymph nodes, 92±6%; bladder, 86±3%; brain, 93±10%; heart, 93±5%; kidneys, 100±2%; liver, 100±7%; lung, 95±8%; muscle, 100±5%; spleen, 96±9%.

FIG. 16 illustrates the in vitro HA-cisplatin release rate at 37° C. for water and PBS solutions. Table 6 shows the associated half life.

6. In Vitro Cell Toxicity

Cell lines were seeded into 96-well plates (5000 cells/well) in DMEM medium supplemented with 5% FBS and 1% penicillin/streptomycin. After 24 hours, cisplatin, cisplatin-HA, or HA was applied (n=12, 7 concentrations), and 72 hours, post-addition, reazurin blue in 10 μL PBS was applied to each well (final concentration of 5 mM). After 4 hours, well fluorescence was measured (λₑₓ 560 nm, λₑₘ 590 nm) (SpectraMax Gemini, Molecular Devices), and the IC₅₀ determined as the midpoint between saline (positive) and cell-free (negative) controls.

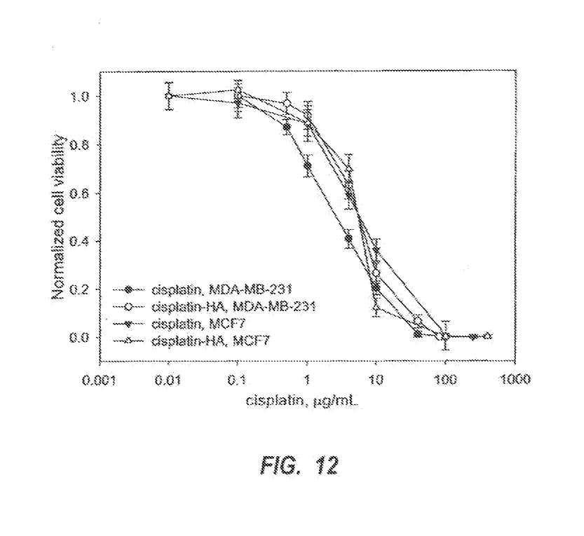

Cisplatin-HA conjugates had similar toxicities to free cisplatin in cell culture. Toxicity was evaluated in the highly metastatic human breast cancer cell lines MCF7 and MDA-MB-231 (FIG. 12). In both cell lines, there was no appreciable difference in toxicity between cisplatin-HA (IC₅₀ 7 μg/mL, cisplatin basis) and, cisplatin (IC₅₀ 7 μg/mL). HA had no toxicity to human cells over the concentration range examined (up to 10 mg/mL, data not shown). Table 7 shows the IC₅₀ values.

7. Atomic Absorption Spectroscopy (“AAS”)

In vitro release samples (n=3) and plasma samples (n=5) were diluted 200-fold and 10-fold, respectively, with 0.1% nitric acid for analysis. Tissue samples (except for lymph nodes) were prepared by digesting 50 mg of tissue in 1.5 mL, of 6.7% nitric acid for 1 hour at 80° C. Lymph nodes were processed similarly using 10 mg of tissue. After digestion, samples were homogenized (Tissue Tearor, BioSpec Products Inc., Bartlesville, Okla.). All samples were centrifuged (17 000×g, 20 minutes), and the supernate used for analysis.

Analysis was performed on a Varian SpectrAA GTA-110 with graphite furnace and partition tubes. Samples (21 μL) were injected using the autosampler, followed by 19 μL of 0.1% nitric acid. Every 10 samples were bracked by calibration standards at 150, 300, and 450 ng/mL, and a quality control sample (150 or 300 ng/mL) every 5 samples. A full calibration curve was prepared from 1 to 450 ng/mL in 0.1% nitric acid (10 concentrations). Cisplatin recovery was determined by spiking tissue blanks with cisplatin or cisplatin-HA (50 μg/g) and processing as above. The furnace program was as follows: ramp 25 to 80° C., hold 2 seconds, ramp to 120° C., hold 1.0 seconds, ramp to 1000° C., hold 5 seconds, ramp to 2700° C., hold 2 seconds, cool to 25° C. over 20 seconds. The graphite partition tube was cleaned every 40 samples by baking at 2800° C. for 7 seconds. Argon was used as the injection and carrier gas.

8. In Vivo Imaging

In order for nanocarriers to deliver anticancer drugs to nano- and micrometastases in the breast loco-regional lymphatics, carriers should drain from the breast area to the diseased lymph nodes. To verify anionic nanocarriers to do, a fluorescent anionic nanocarrier was prepared by coupling 35 kDa hyaluronan with Texas Red hydrazide (AnaSpec, San Jose, Calif.) using EDAC-mediated amide coupling followed by dialysis to remove free dye (0.1% w/w dye/carrier determined spectroscopically). Fluorescent nanoparticles (0.25 μg in 50 μL of saline) were injected subcutaneously beneath the nipple of a xenograft bearing lymphatic metastases.

Fluorescence was measured in 10-nm bandpass segments from 520 to 720 nm, using a cooled CCD camera with autoexposure. Images were spectrally unmixed using the automatic deconvolution tools (Maestro ver. 2.4) to limit skin and intestine autofluorescence resulting from chlorophyll in food.

Lymphatic breast tumor metastasis were induced in nude mice according to the procedure of Chambers and coworkers, who were kind enough to provide the lymphatically metastatic breast tumor cell line MDA-MB-468LN. Nude mice (25-30 g females, Charles River) were anesthetized with pentobarbital (50 mg/kg), and 100 μL of MDA-MB-468LN (10⁷ cells/mL) was injected orthotopically into the left second thoracic mammary fatpad through a small incision later closed with a wound clip. Tumors were palpable after 4-5 weeks (100-300 mm³). Before imaging, mice were anesthetized, and Texas Red-HA (10 mg/mL, in saline; 20 μL) was injected subcutaneously over the left mammary fatpad. The injection area was massaged gently for 5 minutes and fluorescently imaged after 5 and 18 hours (CRI Maestro Flex, CRI Inc., Woburn, Mass.) using a 445- to 490-nm filtered halogen excitation light and a 515-nm longpass emission filter.

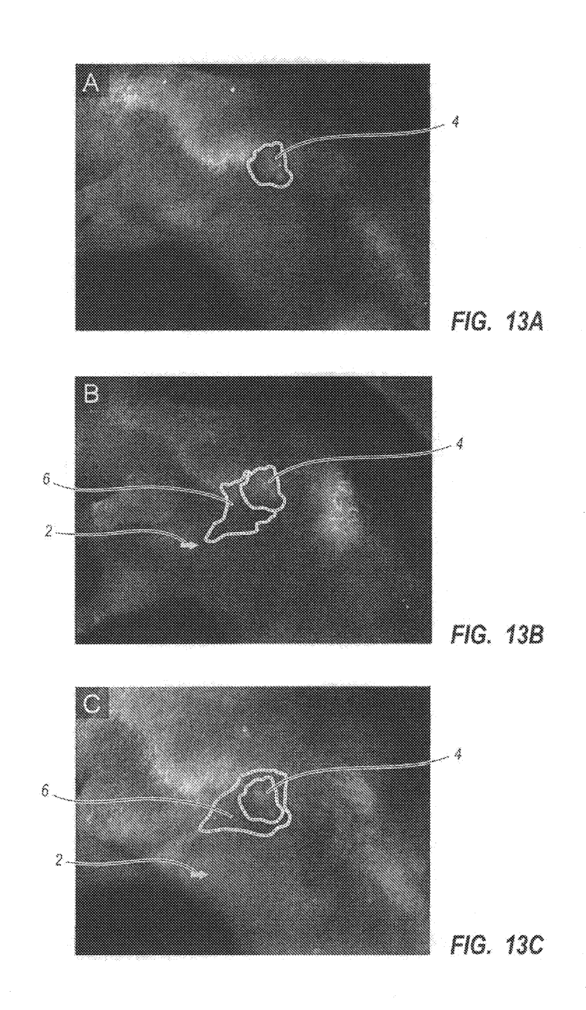

Four hours post injection, fluorescent imaging (λₑₓ 480, λₑₘ 500-720 nm, spectrally deconvoluted) shows that anionic nanoparticles accumulated in a region consistent with the axillary node group (FIG. 13A). At this early time point, much of the carrier is still in close proximity to the injection site, although some trafficking to the axillary lymph node tumor is evident. These images are in deconvoluted, so overlapping GFP and Texas Red signals do not appear yellow. The software includes a colocalization tool the confirmed Texas Red in the area of the tumor. Accumulation of Texas Red-labeled nanocarriers is more apparent in the coronal view (FIG. 13B).

After 18 hours post-injection, the nanocarrier has concentrated in the axillary node surrounding the tumor (FIG. 13C). Of note, no carrier fluorescence appears at the injection site, and there is no apparent spread to the liver, compared against the very high axillary node levels. Obvious areas of carrier localization around the tumor are apparent in the coronal view. Kobayashi et al. observed that cationic PAMAM nanoparticles did not enter lymphatic tumors, but trafficked around them. However, anionic carriers have a much longer residence time, possibly allowing the carrier and its anticancer drug cargo to enter tumors.

FIGS. 13A-13C show the localization of the intralymphatic carrier after subcutaneous injection in nude mice bearing MDA-MB-468 breast lymphatic tumors expressing green fluorescent protein (“GFP”). FIG. 13A shows the breast lymphatic tumor 4 at the time that the mice were subcutaneously injected with Texas Red-HA 6 in the left mammary fat pad. After hours and 18 hours (FIG. 13B and FIG. 13C, respectively), the photographs show that significant HA localized in the draining nodes and co-located with the tumor (GFP-channel in green in color and marked with 4, Texas Red channel in red and marked with 6, the blue arrow 2 is the injection site).

9. Activity

Conjugation of doxorubicin to the nanocarrier did not significantly affect its anticancer activity in breast cancer cells. The 1.0% w/w conjugate was applied to cells, and 72 hours later, cell proliferation was determined using the Alamar blue assay (Invitrogen Corp.). There was no statically significant difference in anticancer activity between the conjugate and free doxorubicin in three aggressive human breast cancer cell lines. Similarly, cisplatin conjugates were applied to breast cancer cells in culture. After 72 hours, cisplatin conjugates were not significantly different from free cisplatin. Overall, conjugation of anticancer drugs to nanocarriers did not decrease the anti-proliferative effect in vitro. This may be due to release of anticancer drugs from nanocarriers into the culture media, followed by uptake as with free drug. However, the microscopy uptake study suggested drugs may have activity despite conjugation to an anionic carrier.

10. Synthetic Schemes

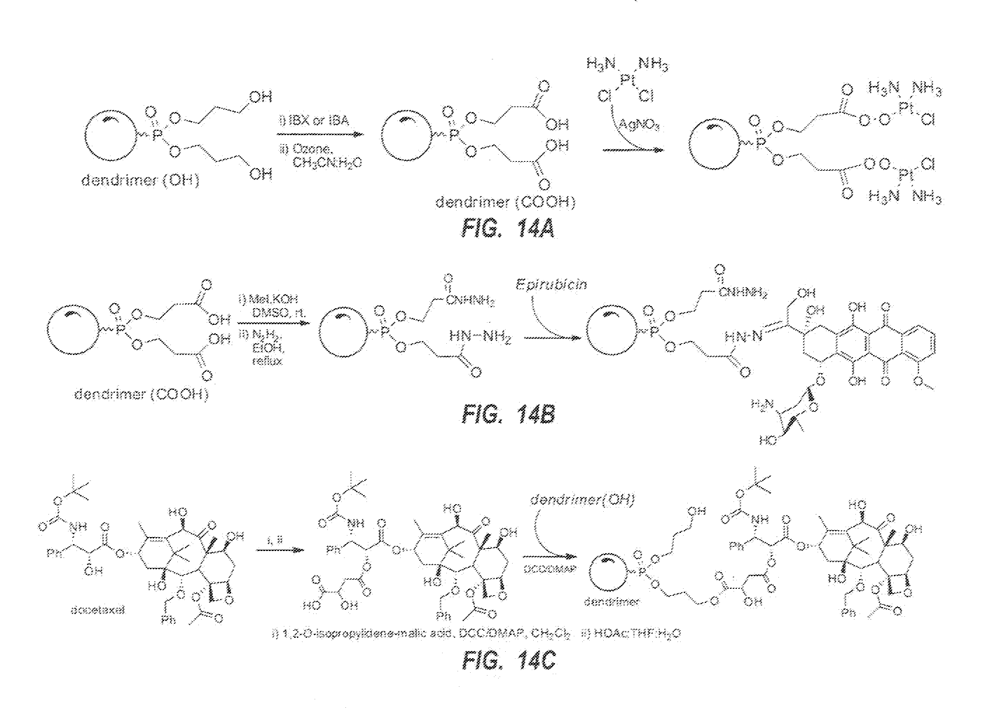

FIG. 14A shows the synthesis of a dendrimer-cisplatin conjugate. The bis-hydroxypropyl phosphate termini of the dendrimer are converted into carboxylic acids, which then form a complex with cisplatin by displacement of the chloride. The resulting complexes will slowly release the potent DNA crosslinker cis-[Pt(NH₃)₂Cl(H₂O)]⁺ at physiological conditions. In preliminary studies, cisplatin conjugates to hyaluronan carriers were formed by displacement of the platinum chloride with a carrier carboxylic acid to form —C(═O)OO—PtCl(NH₃)₂ conjugates. A similar scheme can be used to conjugate cisplatin to the anionic dendrimer carrier. The bis-hydroxypropyl phosphate termini of dendrimers are be converted to carboxylic acids by mild reduction with Dess-Martin periodinane (“DMP”), IBX, or IBA, and ozonation into the carboxylic acid. Alternatively, the hydroxyls can be converted to carboxylic acids by treatment with succinic anhydride/DMAP, although this would extend the termini by four carbons and enlarge the nanocarrier. Complexes will be purified by dialysis or Sephadex, and the drug quantity on nanocarriers will be determined by graphite furnace atomic absorption spectroscopy. The optimum amount of cisplatin conjugation can be between about 10%-50% substitution, more preferably about 20%-40%, and most preferably about 25%-35%. For example, up to 25% w/w conjugation has allowed significant lymphatic uptake of Pt(II)-hyaluronan nanoparticles in vivo.

FIG. 14B shows the synthesis of dendrimer-epirubicin conjugates. The his-hydroxypropyl phosphate termini of the dendrimer are converted into hydrazides, which then form a pH-sensitive hydrazone with the epirubicin. The resulting complexes will release epirubicin in response to decreased pH in endocytic vessels of cells (e.g., tumor cells). Nanocarrier conjugates can greatly increase the amount of anthracyclines accumulated in cancer cells.

The pH-sensitive conjugates are formed using a hydrazone linker between the anthracycline 13-carbonyl and a grafted hydrazide on the nanocarrier. The hydrazide linker was formed by graphing an adipic dihydrazide to the carboxylic acid residues using literature procedures, but a more direct strategy is to convert the nanocarrier hydroxyl termini directly into hydrazides, which will retain the size of the dendrimer. Amine termini would be expected to increase the cationic nature of the carrier, but the low pKₐ of hydrazides (ca. pH 4.5) will minimize this effect extracellularly. In addition, partial conversion of the hydroxyl termini can create enough hydrazides to conjugate the desired drug load. After converting the his-hydroxypropyl phosphate termini of the dendrimer into carboxylic acids, activate and then treat the termini with hydrazine, forming hydrazide termini. The epirubicin conjugate is formed by incubation of the carrier and epirubicin at pH 6.5 to form the reversible hydrazone linker followed by dialysis or Sephadex workup to remove the unconjugated drug. The optimum amount of epirubicin conjugation can be between about 5%-20%, more preferably about 8-15%, and most preferably about 10%-12%.

FIG. 14C shows the synthesis of dendrimer-docetaxel conjugates. The docetaxel is converted into a protected malic acid, and the new carboxylic acid is directly linked with the bis-hydroxypropyl phosphate termini of the dendrimer via an ester linkage. The resulting complex will protect tissues from docetaxel toxicity until conjugates release docetaxel in response to decreased pH in endocytic vessels and endogenous esterases. Docetaxel can be conjugated to the unmodified bis-hydroxypropyl phosphate termini of the dendrimer using by a malic acid linker grafted onto the C2 position of docetaxel using an ester bond. The docetaxel C2-OH can be condensed with 1,2-O-isopropylidene-malic acid, forming a protected malic acid of docetaxel. Deprotection with acetic acid will yield the malic acid, which can be directly coupled onto the hydroxyl arms of the dendrimer. The resulting conjugate can release docetaxel in response to acidic pH or endogenous esterases in acidic vesicles. The expected half-life after injection is expected to be greater than 4 hours, allowing sufficient time for lymphatic uptake based on our preliminary studies of anionic lymphatic nanocarriers. The optimum amount of docetaxel conjugation can be between about 5%-20%, more preferably about 8-15%, and most preferably about 10%-12%.

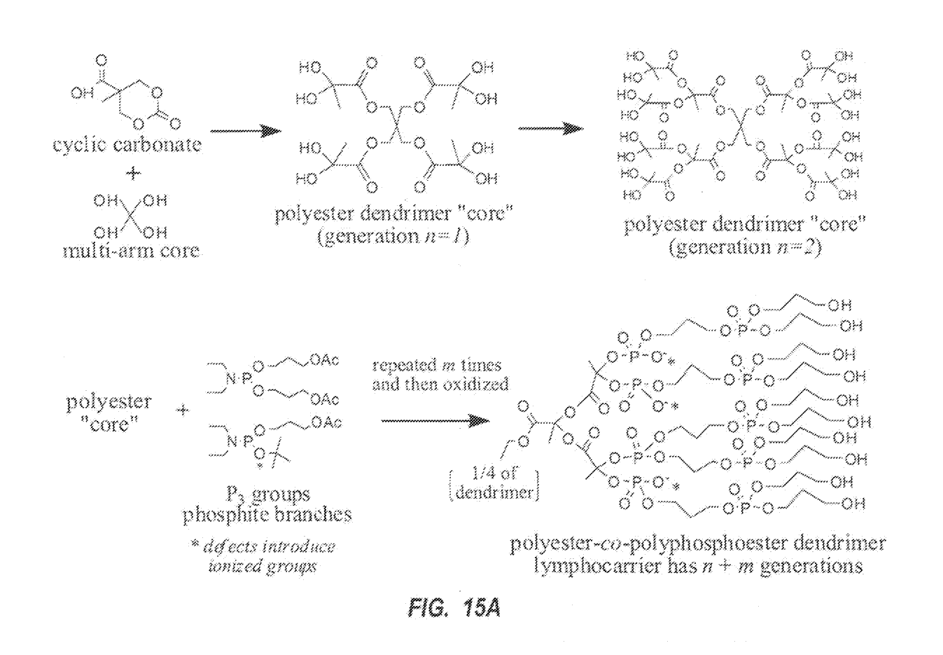

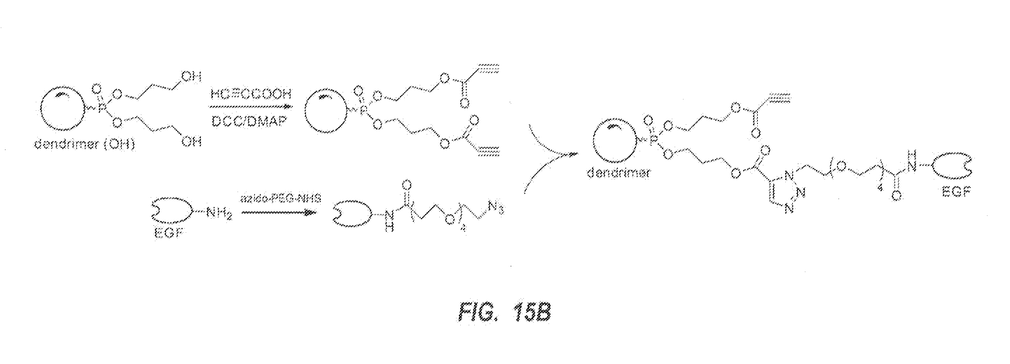

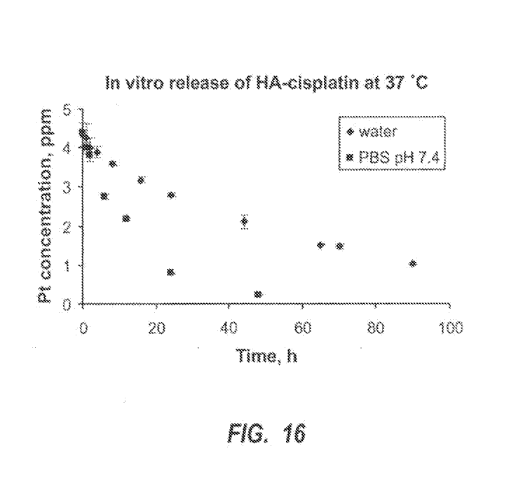

FIG. 15A shows, the synthesis of a dendrimer in accordance with the present invention, and demonstrates an overall synthetic strategy for our library, although we are examining additional “arm” groups and substituents to further improve uptake. From a multi-arm core, we are building a several generation polyester core by carbodiimide coupling, followed by deprotection of the pendant hydroxyls. Additional generations are built from these pendant groups. After 2-4 generations, we switch to a phosphite branching unit (“P3”). Tetrazole is used to couple the P3 branches to the pendant hydroxyls, followed by selenium conversion to the selenophosphate. “Defect” branches (red asterisks) are selectively included to yield ionizable groups when deprotected, thus integrating ionizable groups within the core to limit aggregation and increase solubility. The charge degree can be tailored by varying the ratio of defect branches. After 2-3 generations, the poly-selenophosphoester branches are fully oxidized with organic peroxide, and terminal hydroxyls are converted to carboxylic acids for cisplatin conjugation or hydrazones for doxoruhicin conjugation using standard methods.

Our preliminary studies determined that this dual core strategy can facilitate synthesis. Phosphate groups can be selenium protected, otherwise the phosphate is sufficiently reactive to cleave the arm groups during conjugation; however, in later generations the inner selenophosphates become too hindered by subsequent generations for deprotective oxidation. To overcome these issues we introduce a carboxy ester core, which improves biodegradability and lessens steric crowding during oxidation of the outer phosphate groups. Fully carboxylic acid dendrimers have been developed, but these would not be water-soluble if highly substituted with hydrophobic drugs. We use the anticancer drugs cisplatin and doxorubicin as they are highly effective first-line treatments for multiple cancers including head and neck cancers (“HNC”) and breast cancer.

FIG. 15B shows the synthesis of targeted dendrimers. The bis-hydroxypropyl phosphate termini of the dendrimer are condensed with propiolic acid to form the alkyne-dendrimer. Alternately, the COOH or CONHNH₂ can be condensed with propargyl to form the alkyne-dendrimer. Using a “click” methodology, the dendrimer and the azide of EGF are coupled under aqueous conditions. This strategy can be applied to other targeting agents, e.g., HER2 antibody, EGFR antibody, PSMA, and the like.

The nanoconjugates can target breast cancer cells via the epidermal growth factor receptor (“EGFR”), which is highly overexpressed by aggressive breast cancers with poor prognosis. In addition, EGFR is endocytosed and can be used to internalize nanocarriers conjugated to EGF. Although EGFR is expressed at lower levels by other tissues, passive localization of the nanocarrier to the lymphatics will minimize nonspecific interactions. Unlike systemic nanocarriers, the targeting moiety can improve cell uptake, as physical characteristics alone can localize anionic nanocarriers in the lymph nodes.

Epidermal growth factor (“EGF”) is a 6054-Da protein with lysine residues that may be used to link nanoparticles without decreasing interaction with cells. Since the termini group of the dendrimer is dependent on whether the dendrimer is used for cisplatin (“COOH”), docetaxel (“OH”) or epirubicin (C═ONHNH₂), it is easiest to use a “click” type linker so all chemistries can be done in aqueous solution and as a final step. EGF is functionalized using azido-PEG-NHS ester (Quanta Biodesign Ltd., Powell, Ohio). Alternatively, other targeting aunts are easily added by forming the respective azide. Alkynes can be added to the dendrimer termini using DCC/DMAP chemistry and either propargyl alcohol (COOH termini) or propiolic acid (OH and C═ONHNH₂ termini). The resulting targeted alkyne-dendrimer is mixed with the EGF-azide with a small amount of copper catalyst to form the conjugate, followed by Sephadex or dialysis purification to remove unbound EGF.

11. Tumor Response

Nude nu/nu mice were implanted with 1×10̂6 cells (MDA-MB-468LN human breast cancer cells) into the mammary fatpad, after approximately 4 weeks a 100-200 mm³ tumor had developed the axillary lymph node package. Animals were randomly divided into four treatment groups of five animals each: intravenous saline, s.q. HA, intravenous cisplatin, or s.q. HA-cisplatin. At this point, doses were administered to the animals. In the HA-cisplatin and the HA groups, HA-cisplatin (3.3 mg/kg based on Pt-content) or HA (3.3 mg/kg) was dissolved in 100 mcL saline was injected subcutaneously peritumorally. In the cisplatin and saline groups, cisplatin (3.3 mg/kg, Pt-basis) in 100 mcL saline or Just saline was injected intravenously via the tail vein. In all study groups, a second dose was administered after 1 week. Tumor volumes were measured weekly using calipers and the formula: Volume=0.52×(width²*length). Animals were euthanized when the tumor volume exceeded 1000 mm or if the body score index fell below 2.

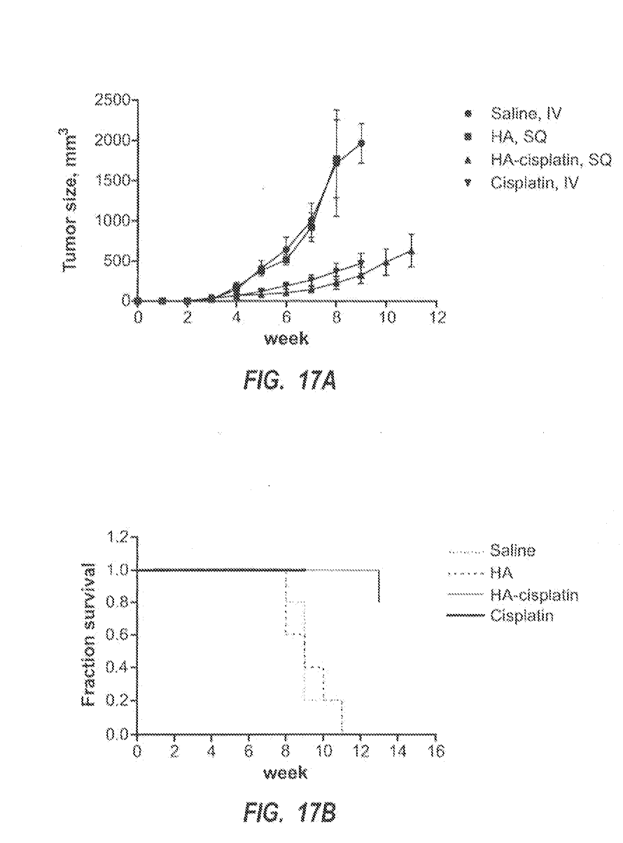

FIGS. 17A-17B show that tumor growth was delayed by HA-cisplatin treatment for 5 weeks compared to negative control group and 2 weeks compared to conventional cisplatin treatment.

The HA-cisplatin formulation delayed tumor growth by 5 weeks compared to cisplatin and no toxicity was observed in the HA-cisplatin treated animals. While it was expected that HA-cisplatin would likely have similar efficacy to intravenous cisplatin, what was unexpected and significant was that HA-cisplatin resulted in longer delays in tumor growth initially, indicating that the higher concentration of drug reaching the tumor allowed it to inhibit tumor growth more effectively during the initial treatment period. This provides convincing supportive evidence that HA-cisplatin subcutaneously given, carries at least the same if not better efficacy than standard cisplatin formulations in this in vivo breast cancer model.

12. Doxorubicin Release

HA was condensed with adipic dihydrazide (“ADH”) at the carboxylic acid group of the gluconic acid in HA. First, ADH and HA (1 eq. of ADH per eq. of HA disaccharides) were mixed with 2 eq. N-(3-Dimethylaminopropyl)-N′-ethylcarbodiimide hydrochloride (“EDAC”) in water adjusted to pH 4.7 with HCl and a final HA concentration of ca. 1% w/w. After 20 minutes, the mixture was dialyzed against water using 13000 MWCO dialysis tubing for 2 days. The product was then conjugated to doxorubicin (“DOX”) in pH 6.5 phosphate buffer (2 eq. HA disaccharide per 1 eq. of DOX) for 24 hours in the dark. The mixture was then dialyzed against pH 6.5 phosphate buffer for 2 days. The final material was lyophilized and reconstituted in saline before use. For release experiments, reconstituted HA-DOX or DOX were placed in dialysis tubing and placed in a bath containing either pH 5, 6.0, or 7.4 phosphate buffer at 37° C. Samples were taken from the dialysis tubing the total drug remaining was determined by HPLC with a fluorescent detector, using a standard calibration curve. The resulting composition was substantially free of any unreacted adipic dihydrazide. The resulting composition was also free of surfactants and solvents.

FIG. 18 shows the release of doxorubicin as a function of pH. The release half-life was found to be 167 hours at pH 7.4, 107 hours at pH 6.0, and 45 hours at pH 5.0.

The HA-DOX provided very sustained release, dependent on the pH of its environment. They may offer substantial benefit, as the interior of solid tumors is known to be hypoxic and to have a reduced pH, thus DOX release from HA-DOX may be accelerated in neoplastic tissues as compared to normal tissues.

To evaluate the efficacy of HA-doxorubicin conjugates injected subcutaneously in the rat breast compared with intravenous doxorubicin, the following experiment was performed. MDA-MB-468LN tumor cells into nude mice as described in [128] and divided into four groups of five animals each. After 3 weeks, HA-DOX was injected peritumorally at 3.3 mg/kg (DOX basis), DOX was injected in saline via the tail vein, HA was injected peritumorally, and saline was injected via the tail vein. Animals were given a second dose at 5 weeks post-implantation.

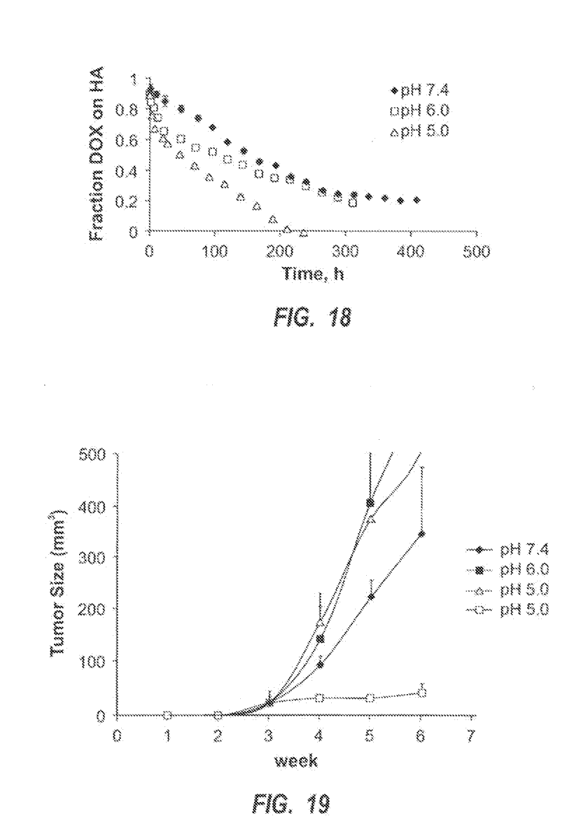

FIG. 19 shows the tumor growth was halted by nanocarrier-DOX treatment after two weekly doses at 3rd and 5th week.

While it was expected that HA-doxorubicin would likely have similar efficacy to intravenous doxorubicin, what was unexpected and significant was that HA-doxorubicin resulted in complete arrest in tumor growth with treatment, indicating that the higher concentration of drug reaching the tumor allowed it to inhibit tumor growth more effectively during the treatment period. This provides convincing supportive evidence that HA-doxorubicin subcutaneously given, is clearly superior in efficacy compared to standard doxorubicin formulations in this in vivo breast cancer model.

HA-DOX substantially retard tumor growth in animals compared to intravenous DOX or the HA and saline controls. After 6 weeks post-implantation, the DOX and HA and saline controls had tumor volumes greater than 300 mm³, whereas the HA-DOX treatment group's tumor were less than 50 mm³. The effectivity was a surprise as the DOX-HA not Only provided effective localized control of tumor growth but also prevent the appearance of new local, regional, or distant growths. HA-DOX animals did not exhibit any toxicity due to dosing. The HA-DOX may provide excellent locoregional control as a “boost” dose to the locoregional lymphatic and tissues of the tumor, and slow drug release may be able to replace the need for many multiple dosings. In addition, the HA-DOX may provide sustained plasma levels of drug compared to intravenous DOX, thus replaced the need for additional systemic therapy. Once again, sustained drug release may reduce the need for repeated dosing. This results were surprising, as the localized chemotherapy was not expected to provide sufficient plasma levels of drug to provide distant tumor control.

13. Phosphoester-Hyaluronan Nanocarriers

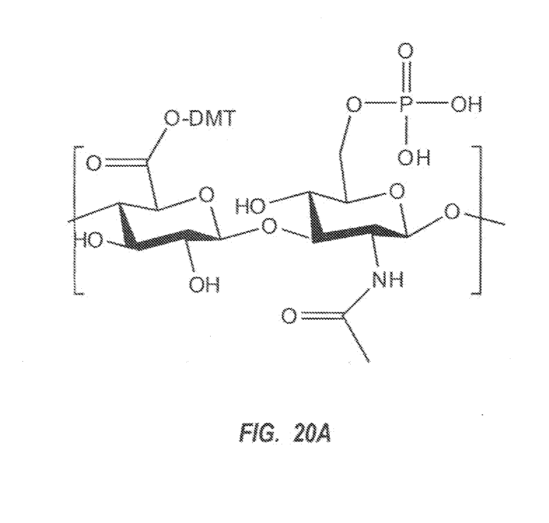

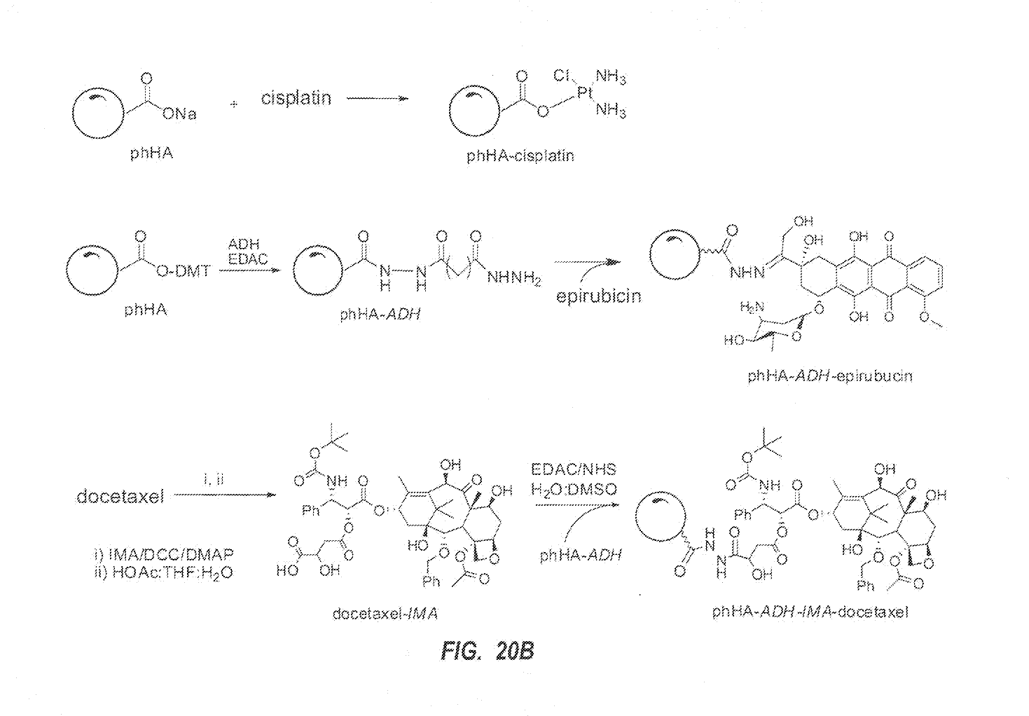

FIG. 20A illustrates a highly water-soluble and biodegradable phosphoester hyaluronan (phHA) nanocarrier. These nanocarrier can be used as described herein.

FIG. 20B illustrates the synthesis of phHA-drug conjugates. The phHA can be functionalized to form labile conjugates (e.g., ester or hydrazone) with anti-cancer drugs cisplatin (top), epirubicin (middle), and docetaxel (bottom). The resulting complexes will protect tissues from toxicity until conjugates release drugs in response to decreased pH in endocytic vessels and endogenous esterases. Dephosphoration of phHA by AP in the lymphatics will promote conjugate accumulation and sustained release of drugs within the lymphatics.

The carboxyl groups of HA (30-50 kDa, corresponding to 10-20 nm can be protected (e.g., with DMT added using NMM/CDMT). The primary alcohol of HA can be converted to a phosphoester (e.g., with 1:1 aq. H₃PO₄ and perrhenic acid). For conjugation with cisplatin, the DMT may be removed from phHA with acid giving the carboxylic acid. For epirubicin and docetaxel, DMT may be displaced with a strong nucleophile such as adipic dihydrazide allowing formation of the pH-sensitive labile hydrazone with drugs.

Sodium phHA can be conjugated cisplatin using the same procedure as in preliminary studies with HA, by overnight reaction with cisplatin (20-40% w/w), and purified by dialysis against water. The degree of conjugation can be determined by graphite-furnace AAS.

Anthracyclines (doxorubicin, epirubicin, and daunorubicin) can be used to form pH-sensitive conjugates of the structurally similar doxoruhicin, using a hydrazone linker between the anthracycline 13-carbonyl and a grafted hydrazide on HA. The hydrazide phHA (described above) can be conjugated to epirubicin by incubation at pH 5, followed by dialysis purification.

Taxanes including docetaxel and paclitaxel can now be prepared into a polysorbate-free formulation. This can be accomplished by forming esters off the C7 carbon that couple with the nanocarrier, with no detriment on anti-cancer activity. Docetaxel can be conjugated to the COOH of the phHA using a labile malic acid linker grafted onto the C2 position of docetaxel.

14. In Vivo Efficacy of HA-Cisplatin

The in vivo efficacy of HA-cisplatin was studied. All treatment began once animal HNSCC tumors reached 100 mm3 volume. Treatment commenced for 3 weeks. All animals were given 5×10̂5 MDA-1986 cells in the buccal mucosa of the left cheek (which is a novel orthotopic HNSCC model I developed this year) and tumors develop in 2-3 weeks.

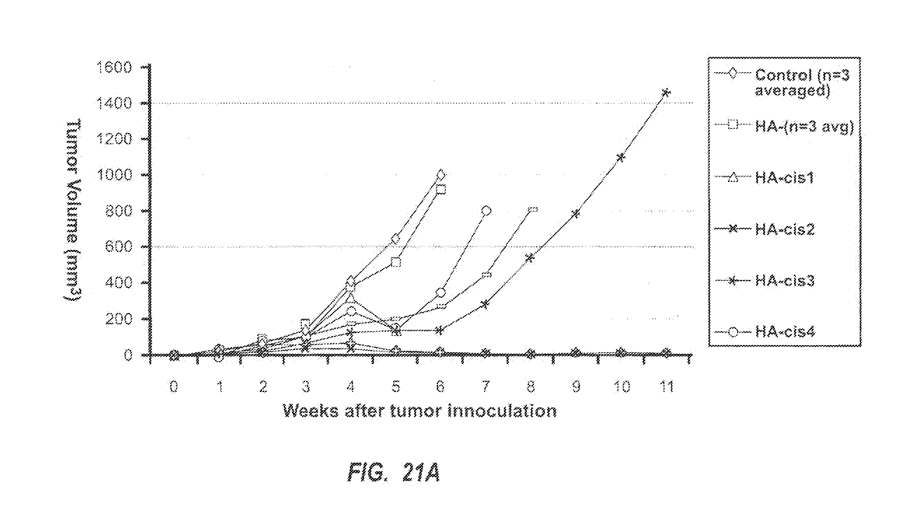

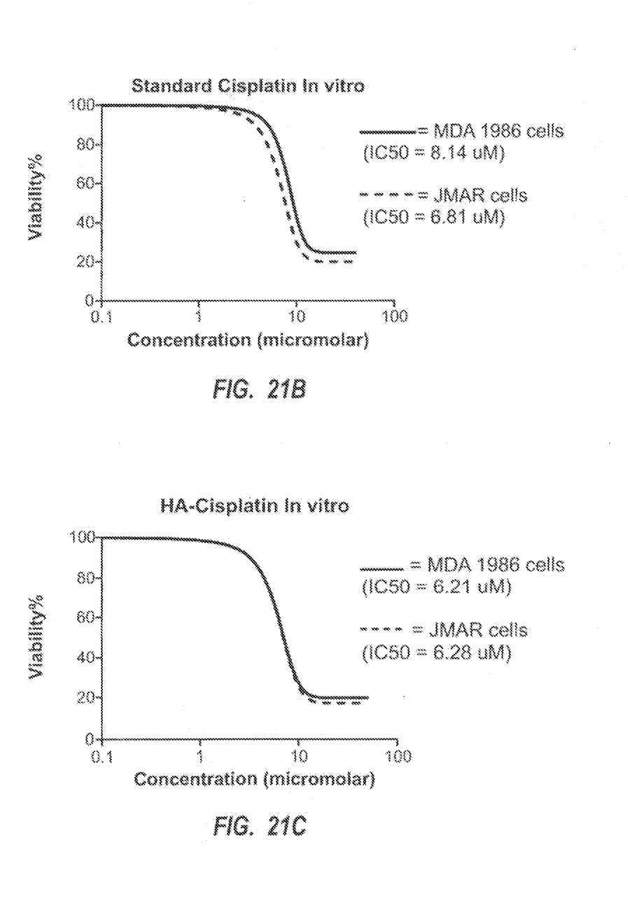

FIG. 21A includes efficacy graph of Nu/Nu female athymic nude mice treated with HA carrier by itself (subcutaneous), control (saline subcutaneous), 5 mice with HA-cisplatin weekly dose of 3.3 mg/kg subcutaneous for 3 weeks, and 3 mice with intravenous cisplatin (weekly dose of 3.3 mg/kg intravenous for 3 weeks). This graph shows that HA-cisplatin delayed tumor growth in all 5 animals, 2 of which (HA-cis 3 and HA-cis 4) were similar to the rate at which i.v., cisplatin (control) delayed growth, by about 3 weeks. Whereas in the other 3 animals, 2 animals had a complete response to therapy and the third (HA-cis 1) had a partial response to therapy before it was sacrificed. Overall, this shows an improved efficacy over standard intravenous cisplatin in treating head and neck squamous cell cancer in vivo.

To evaluate the anticancer, specifically antiproliferative, effects of HA-cisplatin compared to standard CDDP in vitro in two human head and neck squamous cell carcinoma cell lines (JMAR and MDA-1986) a standard MTS assay was performed as per the manufacturer's specified protocol.

Once 75% confluent, cells were trypsinized (0.25% trypsin), counted and plated in 96-well microtiter plates (Costar, Cambridge, Mass., USA) (1×103 cells/well) in 100 μL of growth media. After an overnight attachment period, cells were exposed to varying concentration of KU135 and 17-AAG, alone and in combination for 3 days. All studies were performed in triplicate and repeated at least three times independently. After the 3-d treatment period, the number of viable cells was determined using a colorimetric Cell Proliferation assay (CellTiter96 Aqueous Nonradioactive Cell Proliferation assay; Promega, Madison Wis., USA), which measures the bioreduction of the MTS (3-[4,5-dimethylthiazol-2yl]-5-[3-carboxymethoxyphenyl]-2-[4-sulfophenyl]-2H tetrazolium) by dehydrogenase enzymes of metabolically active cells into soluble formazon product, in the presence of the electron coupling reagent PMS (phenazine methosulfate). To perform the assay, 20 μL of combined MTS/PMS solution containing 2 mg/mL, MTS and 150 μM PMS in buffer (0.2 g/L KCl, 8.0 g/L, NaCl, 0.2 g/L KH2PO4, 1.15 g/L, 133 mg/mL CaCl2-2H2O, 100 mg/mL, MgCl2.6H2O, pH 7.35) was added to each well and then after 3 hours of incubation at 37° C. in a humidified 5% CO2 atmosphere, absorbance was measured at 490 nm in microplate reader. Triplicate wells with predetermined cell numbers were subjected to the above assay in parallel with the test samples to normalize the absorbance readings; this also provided internal confirmation that the assay was linear over the range of absorbance and cell numbers measured. Data was plotted as a function of % viability from controls (cell viability) vs. drug concentration (x-axis). The concentration of drug at which 50% of cells were inhibited from growth (IC₅₀ level) was determined as the point of inflection on a standard absorbance-concentration curve.

FIG. 21B demonstrates percent cell viability versus drug concentration curves for standard cisplatin against both cell lines after 72 hours drug treatment at varying concentrations of drug ranging from 10 nM to 100 micromolar concentrations. FIG. 21C demonstrates percent cell viability versus drug concentration curves for HA-cisplatin against both cell lines after 72 hours drug treatment at varying concentrations of drug ranging from 10 nM to 100 micromolar concentrations. IC₅₀ concentrations were calculated from these graphs as the point of inflection where 50% Of cell growth is inhibited. Of note, there is no significant difference in either cell line between IC₅₀ levels for CDDP and HA-cisplatin indicating that in vitro, HA conjugation does not adversely effect cisplatin's ability to inhibit cancer cell growth and viability.

15. In Vivo Release of Doxorubicin

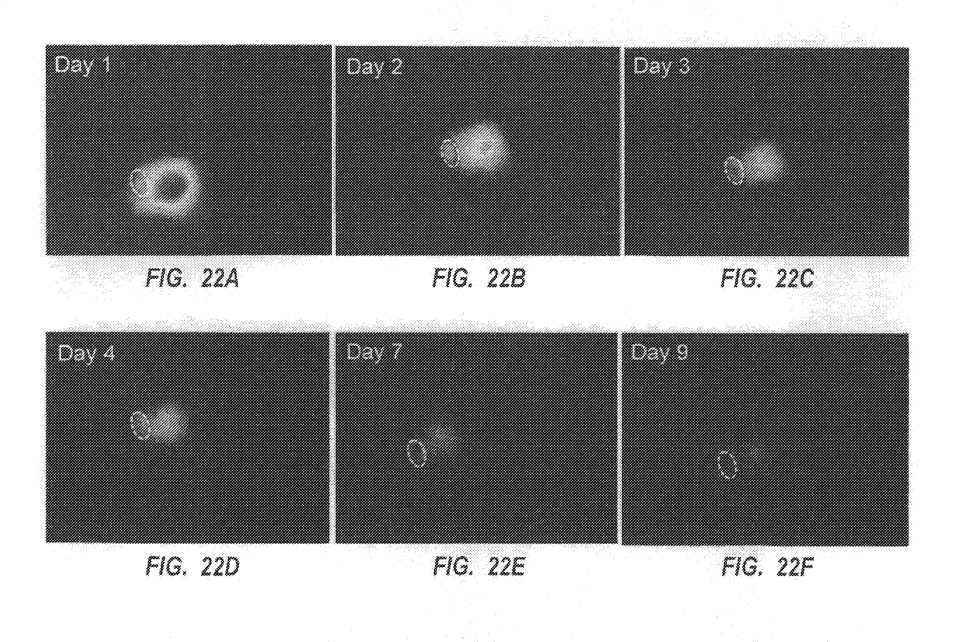

FIGS. 22A-22F are photographs showing the distribution of HA-doxorubicin after a single injection in the right mammary fat pad of a rat. Doxorubicin has innate fluorescence and the distribution and longevity of the drug-carrier conjugate can been well observed in this timed evaluation. Of note the bulk of drug-carrier is transported to the axillary lymph nodes where is slowly releases drug over a 9 day interval with still some residual activity even after 9 days. The oval marks the injection site in the breast and the darkest concentration (red) is in the axilla.

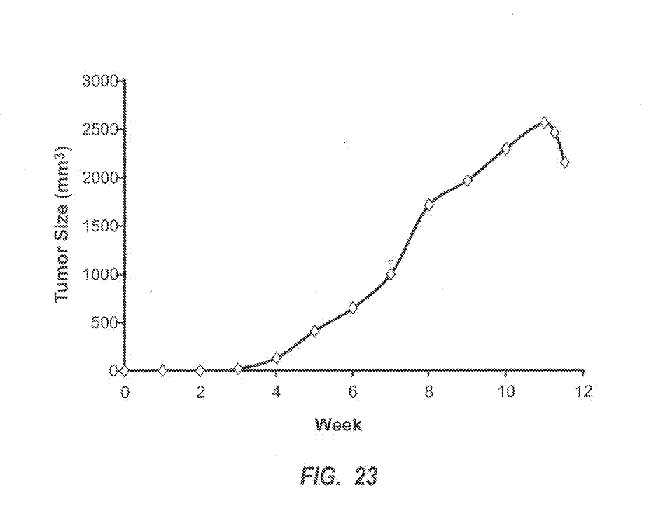

FIG. 23 is a graph showing tumor response even after a single late term peritumoral HA-Doxorubicin treatment in a considerably advanced breast cancer tumor in vivo.

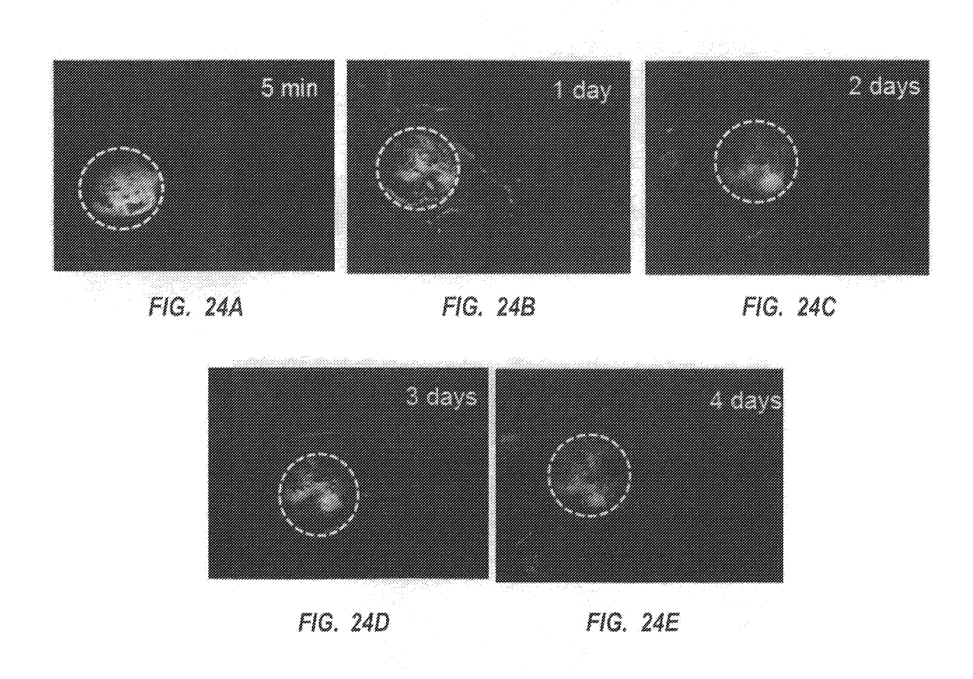

FIGS. 24A-24E are photographs of in vivo trafficking of HA-doxorubicin as visualized on an Maestro multichannel fluorescent imaging system. There is nice uptake of drug and carrier into the locoregional tissues and lymph nodes of the rat breast, which stays well in the lymphatic's even 4 days post-injection.

16. Combination Therapies Involving HA-Dox and HA-Cis.

In this example, a combination therapy of doxorubicin and cisplatin when conjugated to hyaluronan was investigated and the efficacy was compared with standard drugs.

Cell Culture

The lymphatically active metastatic breast cancer cell line MDA-MB-468LN obtained as a gift from Dr. Chambers and coworkers was maintained in modified Eagle's medium alpha (Sigma-Aldrich, St. Louis. MO), supplemented with 10% fetal bovine serum (FBS), 1% L-glutamine and 0.4 mg/mL. G418 (Sigma Aldrich). Adherent monolayer cultures were maintained in T-75 culture flasks and incubated at 37° C. with 5% CO₂ until they achieved 85% confluency. The cells were trypsinized using 0.25% trypsin (Sigma Aldrich) and passaged into T-75 flasks at a density of 1×10⁶ cells. On experiment days, cells were trypsinized and counted via hemocytometer to determine the number of viable cells.

In Vivo Tumor Model and Treatment

Lymphatic breast tumor metastasis was induced in nude mice according to the procedure of Vantyghem et al., A new model for lymphatic metastasis: development of a variant of the MDA-MB-468 human breast cancer cell line that aggressively metastasizes to lymph nodes, Clin Exp Metastasis. 22(4) 351-361, (2005), who provided the lymphatically metastatic breast tumor cell line MDA-MB-468LN. MDA-MB-468LN breast cancer cells were prepared in a 1×PBS solution at a concentration of 1×10⁶ cells/100 μL. Cells (100 μL) were injected under isoflurane anesthesia into the right first breast mound (abdominal mammary fat pad) of 4-6 week old female Nu/Nu mice using a 25 G needle (20-25 g, Charles River Laboratories, Wilmington, Mass.). Tumor size was measured 3 times weekly using a digital caliper and confirmed by two separate observers. Tumor volume was calculated using the following equation: tumor volume (mm³)=(π/6)×(width)²×length. When tumors reached a minimum volume of 30 mm³, mice were randomized into control (PBS or HA) or one of four combination treatment groups [50% Maximum Tolerated Dose (MTD) Doxotubicin+50% MTD Cisplatin (Dox-Cis 50), 75% MTD Doxorubicin+75% MTD Cisplatin (Dox-Cis 75), 50% MTD HA-Doxorubicin+50% MTD HA-Cisplatin (HA-Dox-Cis 50), and 75% MTD HA-Doxorubicin+75% MTD HA-Cisplatin (HA-Dox-Cis 75). Pharmaceutical grade Doxorubicin and cisplatin were used for the standard treatment groups as well as to create the nanocarrier formulation as previously described. The HA control and HA treatment groups were administered subcutaneously (s.q.) 1-3 mm away from the site of tumor implantation and the PBS control and standard treatment groups were administered intraperitoneally (i.p.). The MTD level reported in mice for doxorubicin is 8-10 mg/kg/weekly i.p. dose and for cisplatin is approximately 10 mg/kg/weekly i.p. dose. All treatments were given 1×/week for a total of 3 weeks and mice were monitored for an additional 9 weeks upon completion of treatment (total study period of 12 weeks). Mice were euthanized prior to completion of the experiment if the tumor reached greater than 20 mm in diameter, if weight loss was significant, or if body score markedly deteriorated.

Pathology Studies

Two NU/Nu mice from each of the treatment groups were euthanized 1 week after completion of treatment (week 4) and an additional 2 mice from each group were euthanized at the completion of the study for histologic analysis of tumor, organ, and injection sites. The tumor site with surrounding skin, heart, lungs, brain, bilateral kidneys, spleen, liver, bone marrow from spine and femur, and ipsilateral (right) as well as contralateral (left) axillary lymph nodes were harvested intact from the mice and stored in 10% formalin solution for fixation overnight prior to slide mounting. Mounting using hematoxylin and eosin staining was conducted by the University of Kansas Medical Center Department of Pathology (Kansas City, Kans.) and histologic examination was performed by a blinded board-certified pathologist. Slide images were obtained using Aperio version 10.0 software (Aperio Technologies, Inc., Vista, Calif.).

Statistical Analysis

Comparisons of differences between two or more means were determined by Student's unpaired t-test (2 means) and Fisher's exact test. Multivariate analysis was performed by 2-way ANOVA followed by Duncan's multiple range test (2+ means) and Bonferroni post-hoc testing using a statistical analysis software package (SPSS version 17.0; SPSS Inc, Chicago, Ill.). Significance was defined for p<0.05.

Results

In Vivo Efficacy Analysis

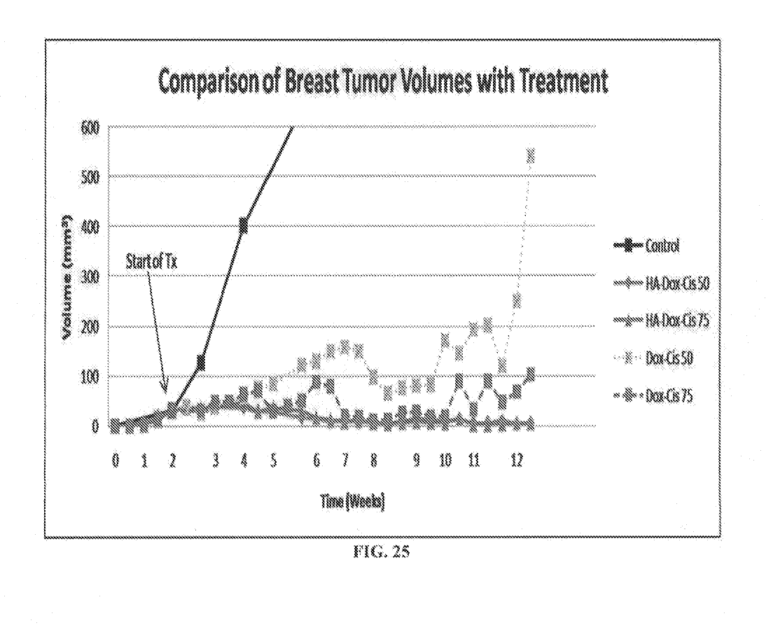

To examine the efficacy of HA-doxorubicin and HA-cisplatin in vivo, tumor volumes were monitored in the mice and confirmed by histologic analysis. The control animals (PBS and HA-only) demonstrated a standard tumor growth curve with tumor volumes exceeding 1200 mm³ by six weeks post inoculation (FIG. 25). There was no difference noted in tumor growth curves between PBS controls and HA (carrier only) control animals, confirming that HA by itself has no direct anti-tumor activity. These groups were combined as a composite control curve (FIG. 25). Of the experimental groups, HA-Dox-Cis 75 was noted to have the best overall efficacy. With 100% of the mice showing response to treatment and 7 of 8 mice (87.5%) having a complete response (“CR”) and the remaining mouse having a partial response (“PR”) with 87% reduction in tumor volume (FIG. 25). The second best group for efficacy was the HA-Dox-Cis 50 group, where 7 of 8 mice (87.5%) had a significant response to treatment (5 CRs and 2 PRs), with the remaining mouse having stable disease (“SD”), FIG. 25. Alternatively, in the standard treatment groups at comparison MTD levels, the Dox-Cis 75 group had only 2 of 8 (25%) animals with a partial response to treatment with the remaining 6 animals having either stable disease (N=3) or progressive disease (N=3). Finally, in the Dox-Cis 50 group, there was only 1 PR (12.5% response rate), one animal with stable disease, with the remaining 6 animals (75%) having progressive disease (PD, FIG. 25). Of note, there were no complete responders noted in either of the standard treatment groups and in the HA-Dox-Cis 75 mice, all CRs were true pathologic complete responses. An overall comparison of all 4 treatment groups using a multivariate analysis was noted to be statistically significant at p<0.0001 and When breaking this down to compare individual groups, the response rate among the HA-Dox-Cis 50 group compared to the standard 50 group and the standard 75 group was noted to be statistically significant (p=0.0004 and p=0.005, respectively). Conversely, comparing the HA-Dox-Cis 75 group to the standard 50 and 75 groups was also noted to be statistically significant (p<0.0001 and p=0.0003, respectively). Of note, comparison between the 2 closes levels of the standard treatment was not noted to be statistically significant (p=0.27).

Pathologic Analysis

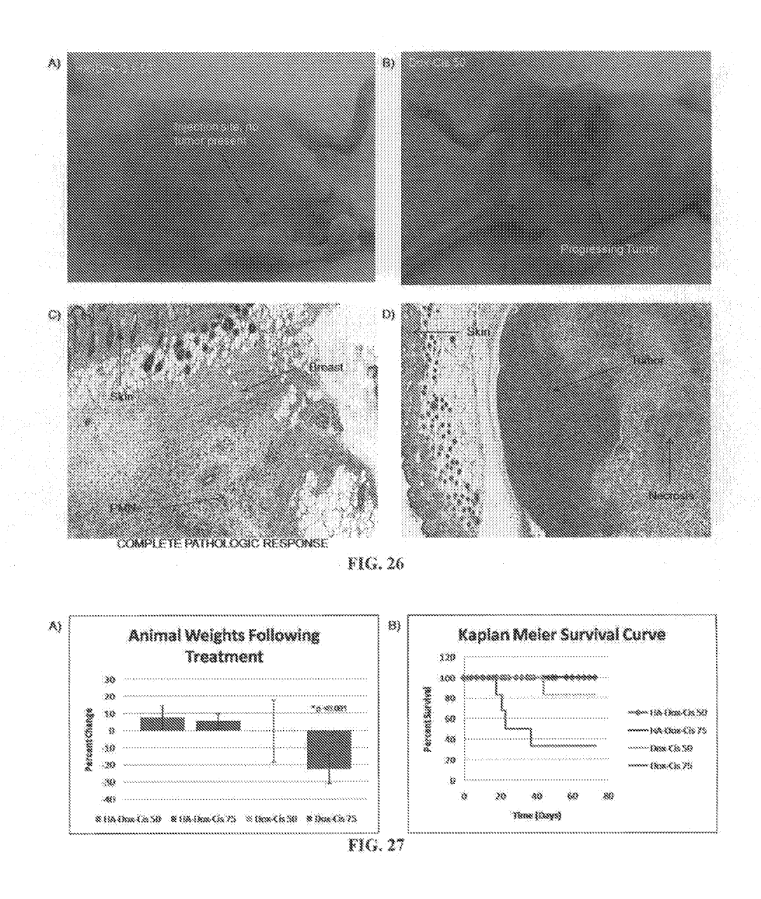

In the complete responders of both the HA-Dox-Cis 50 and the HA-Dox-Cis 75 treatment groups, no visible tumor could be seen grossly (FIG. 26A) compared to the visible tumors in the standard Dox-Cis 50 and Dox-Cis 75 groups (FIG. 26B). To confirm the significance of these findings, the tumor sites, as well as bilateral axillary lymph nodes, heart muscle, and kidneys were examined histologically for all treatment groups. The tumor site and lymph nodes were examined for evidence of residual microscopic cancer disease and the heart and kidneys were examined for evidence of systemic toxicity. Upon histological examination, both HA treatment dosing groups showed fibrosis and neutrophil infiltration but no histologic evidence of residual tumor at the tumor site (FIG. 26C) compared to the standard treatment at both doses, which had residual tumor with associated central necrosis (FIG. 26D). Additionally, there was no evidence of lymph node metastases present in any of the treated animals while over 80% of controls developed metastatic disease to lymph nodes and lungs. Finally, evaluating organ and bone-marrow toxicity, there was no evidence with the short term-dosing used in this study of any histologic toxicity at the injection site, bone marrow, heart, or kidney in any of the treated groups. Systemic disease was however noted histologically as spinal metastases in one of the mice at the 50% MTD systemic Dox-Cis combination whereas none of the mice in the HA-groups demonstrated any systemic disease.

In Vivo Toxicity Analysis

In addition to histologic toxicity, all mice were evaluated for signs of weight loss or deterioration in body conditioning score as a clinical sign of toxicity. All of the animals in both HA groups had no sustained weight loss or deterioration in body score throughout the study. Also, there was no significant difference noted between either HA dosing group with respect to weight loss (p=0.49:17). In comparing the weight loss profiles of the HA groups to dose-matched standard drug combinations, it was noted that there was no weight loss noted in the standard 50% group as well, however, there was an average weight loss of 23% from baseline in the animals from the Dox-Cis 75 group, which was noted to be statistically significant (p<0.001) (FIG. 27A). It should be noted, though, that the HA-Dox-Cis-75 group did demonstrate some weight loss (average of 10%) while receiving the 3 weeks of treatment, however this effect was transient with all mice returning to their baseline weights within 10 days after completion of treatment. This effect was permanent in the standard groups with deterioration in body score requiring early euthanasia clue to this toxicity, particularly in the Dox-Cis-75 group, where 5 animals were sacrificed for clinical toxicity prior to completion of the study (FIG. 27B).

### DISCUSSION

The data demonstrated that the HA-combination generated less locoregional and systemic toxicity than standard systemic agents at similar dose levels and that a reduced dose of each drug could be administered to achieve similar efficacy. This would have significant advantage clinically if lower doses can be administered in an effort to avoid dose-limiting toxicities of these agents. Lower doses of each drug in combination given via the nanoconjugate peritumoral route achieved the same efficacy as higher doses required when given systemically, suggesting a possible synergistic effect in combination when combined to the nanocarrier. With respect to timing and delivery, each drug is injected individually in the subcutaneous peritumoral area one immediately following the other. If there is extensive regional lymph node involvement which could obstruct the lymphatics, it would be possible to inject the drug just proximal as well as distal to the tumor mass to ensure adequate uptake in the entire lymphatic basin. In terms of the mechanism of this systemic effect, once the HA is cleaved in the lymphatics or peritumorally by the enzyme hyaluronidase which is present in lymph, the free drug can either interact locally at the tumor cell by diffusion or active transport into the cell or will be transported due to its smaller size into the systemic circulation where it will achieve therapeutic systemic levels. The difference between this delivery and intravenous infusion therapy is that the cleavage rate of free drug off the carrier provides a slow, sustained-release of drug with a lower Cₘₐₓ but achieves equivalent plasma AUC levels over time allowing the drug to be effectively therapeutic to systemic metastases as well. Systemic absorption was measured in the nanoconjugates individually in previous studies of these compounds and compared to standard agents. Those studies demonstrated comparable levels of systemic penetration via equivalent plasma AUC levels. Intratumoral as well as lymphatic levels of HA-cisplatin compared to systemic cisplatin were also measured demonstrating significantly increased levels of cisplatin in the tumor and lymphatic tissues in the HA-Cis group compared to systemically delivered cisplatin.

While previous studies have demonstrated improved efficacy and pharmokinetic profiles of nanoconjugated chemotherapeutics as single agents in vivo, the use of combination therapy more closely approximates treatment of breast cancer clinically. Systemic chemotherapeutic agents are often administered in combination due to synergistic effects. Therefore, combination of two nanoconjugated agents in vivo would be expected to further enhance this synergy. Although individual uptake of each drug was not measured intra-tumorally in the combination therapy, based on the dramatically improved efficacy of the combination nanoconjugated agents compared to systemic agents in combination as well as the previously published single agent data, it stands to reason that uptake of these agents is improved. Also, due to the reduced toxicity profile of this delivery system, both nanoconjugated agents can be delivered simultaneously, allowing for increased tumor targeting. In the study, half of the animals in both the 50% and 70% HA-Dox-Cis treatment arms were given both injections at the same site peritumorally while the other half of the animals received each injection on opposite sides of the tumor. NO difference in tumor response was noted between the difference in injection sites.

The results in this study demonstrated that in combination, HA-Dox-Cis was able to generate a complete pathological response in a majority of animals treated even at only 50% of the MTD levels of the standard doxorubicin and cisplatin combination. When this dose was increased to 75% MTD, the HA-Dox-Cis group developed a complete pathologic response in 87.5% of animals treated with the remaining animal having a partial response with 87% tumor reduction. Comparatively, neither of the standard dosing groups had any complete responders, indicating a significantly improved efficacy for the nanocarrier delivered drug combination even at half of the standard dose of current therapy.

With regards to toxicity, the standard treatment at 75% MTD of doxorubicin and cisplatin resulted in significant morbidity and mortality, with 67% of the mice requiring euthanasia prior to study completion due to significant clinical toxicity as evidenced by decreased body score and long-term weight loss. Alternatively, this was not seen in either HA group, although a transient 10% weight loss was noted in the 75% MTD HA group during the treatments which resolved spontaneously. From a histologic standpoint, no evidence of cardiac or renal toxicity was noted in any of the groups, although cardiac toxicity is due to a cumulative dose of doxorubicin and this cumulative effect was not likely achieved with only 3 doses of drug given. Furthermore, differences in renal toxicity may not have been observed in this small group either when only three doses of drug are given, all at 75% or less of their maximum clinical dose. Further investigation with longer follow-up and longer dosing regimens will provide more insight regarding chronic toxicity of the HA-combinational treatment.

Overall, it was concluded that based on this study, nanoconjugated combination therapy with doxorubicin and cisplatin exhibited potent anticancer activity against a locally advanced breast cancer orthotopic murine model in vivo. These data indicate that this combination therapy has improved efficacy (especially locoregionally) with decreased clinical toxicity compared to standard dosing of doxorubicin and cisplatin combinational therapy. The limitations of this study include a small sample size for each group as well as a short (3-week) duration of therapy. Despite these limitations, there was enough of an improvement in efficacy and toxicity with the HA-Dox-Cis at all dosing levels over standard therapy to demonstrate statistical significance.

As this system uniquely targets and boosts drug delivery to the primary tumor, lymphatics, and locoregional tumor bearing tissues, it is uniquely suited for patients who have extensive regional nodal disease. One benefit of using the nanoconjugate is that its sustained release kinetics provide for a lower (less toxic) Cₘₐₓ level in the plasma. It is expected that HA-doxorubicin, therefore, would have excellent efficacy on systemic metastatic disease, which these patients undoubtedly harbor. Clinical use of this nanodelivery for doxorubicin would provide the opportunity to evaluate the added benefit of the locoregional boost on the primary tumor and lymph nodes at the time of surgery and axillary lymphadenectomy as well as the effect on any known systemic metastatic disease and be able to compare this effect to standard systemic therapy alone. Treatment with the nanoconjugate should reduce the tumor burden and lymphatic disease prior to surgical resection in hopes to prevent future recurrence or in patients who have locoregional recurrence and have failed traditional systemic agents or are limited in the administration of these agents to due cumulative dose toxicity. In patients with known concomitant systemic disease, as these agents have systemic penetration, they could be effective at targeting the systemic disease or could provide a useful adjunct to traditional systemic therapy, allowing for a reduced dose of the systemic agent. These data provide a solid foundation for further translation of this delivery system toward a wide range of clinical applications where there may be need for novel treatment strategies that carry less toxicity and morbidity to patients.

18. Synthesis of HA-Docetaxel:

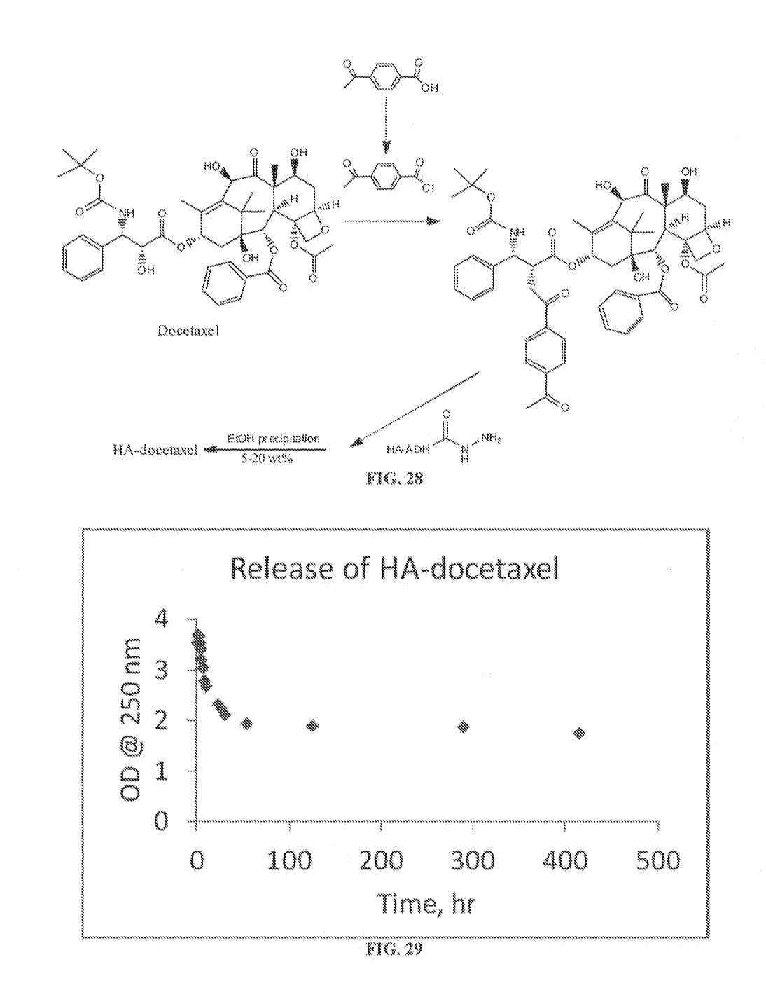

Direct conjugation of drugs to HA is inefficient due to the steric hindrance of the polysaccharide backbone and low reactivity of the carboxylate group. HA was derivatized with adipic acid dihydrazide, according to the procedure of Luo et al. Briefly, HA (200 mg, 35 kDa) was dissolved in 40 mL ddH₂O with ADH (436 mg) and 1-ethyl-3-[3-(dimethylamino)-propyl]carbodiimide (EDCl, 48 mg). The solution pH was adjusted to 4.75 with 1 N HCl, and checked again after 15 minutes. The reaction was quenched by addition of 0.1 N NaOH to pH 7.0. The resulting solutions were dialyzed against ddH₂O for two days with bath changes every 12 hours. After dialysis, the product was filtered (0.2 μm PS membrane, Millipore), and lyophilized. The degree of substitution was determined to be 25% wt/wt by ¹H NMR in D₂O using the ratio of ADH methylene protons to HA acetyl methyl protons. Conjugation of docetaxel (80 mg) to HA-ADH (300 mg) was accomplished using a highly reactive benzoyl chloride linker (50 mg) by formation of a hydrazone between the ketone of docetaxel-linker and the hydrazide side chain of HA-ADH in 50:50 DMSO:H₂O for 15 hours. The HA-docetaxel conjugate was purified using EtOH precipitation in 90:10 EtOH:H₂O (twice) and 100% EtOH (three times). The degree of conjugation was determined to be 4.7% wt/wt using ¹H NMR in D₂O. In order to increase the loading degree, approximately 2-fold extra docetaxel-linker was utilized (180 mg) to react with HA-ADH (300 mg). The resulting degree of substitution was determined to be 19.5% wt/wt using ¹H NMR in D₂O. The scheme is illustrated in FIG. 28. The size was about 15-20 nm; 28.6-kDa HA was used in the synthesis.

19. In Vitro Drug Release of HA-Docetaxel:

The release half-life of docetaxel of the HA backbone was determined to be 55 hours at pH 7.4 using an UV spectrophotometer at a wavelength of 250 nm. As shown in FIG. 29, the release kinetics exhibits a biphasic profile with a relatively rapid release in the initial 10 hours. The slow release during 50-400 hours is likely due to the formation of a hydrophobic domain within the conjugate resulting in the hindered hydrazone bond breakage between the drug and the polymer backbond. The extended half-life at physiological pH was consistent with the sustained release characteristics of HA-docetaxel conjugates.

20. Combination Therapies Involving HA-Dox and HA-Cis and HA-Docetaxel

This example involved an experiment which examined the efficacy of combination treatments with three standard systemic chemotherapeutic agents (docetaxel, cisplatin, and doxorubicin) versus their hyaluronic acid conjugated forms (HA-doxorubin, HA-cisplatin, and HA-docetaxel) in an aggressive, lymphatically metastatic triple negative (ER-, PR-, Her2nu-) breast cancer cell line (MDA-MB-468LN). Mice were injected with approximately 3 million cells in the second right mammary fat pad. Mice were included in the study upon tumor width and length dimensions of >4×4 mm or >5.5 mm in any one direction. Efficacy of single agent treatments was previously completed or concurrently determined. Dosing levels were established based on previously determined individual maximum tolerated doses (“MTD”) with each individual standard compound and set at either 50% or 75% MTD for this study. Therefore, the treatment arms included:

(1) Control (saline injection only)

(2) Standard docetaxel, cisplatin, and doxorubicin, each at 50% MTD (15, 5, and 4 mg/kg/dose, respectively)—Subsequently referred to as the “50% triple standard” arm

(3) Standard docetaxel, cisplatin, and doxorubicin, each at 75% MTD (22.5, 7.5, and 6 mg/kg/dose, respectively)—Subsequently referred to as the “75% triple standard” arm

(4) HA-docetaxel, HA-cisplatin, and HA-doxorubicin, each at 50% MTD (15, 5, and 4 mg/kg/dose, respectively)—Subsequently referred to as the “50% HA triple” arm

(5) A-docetaxel, HA-cisplatin, and HA-doxorubicin, each at 75% MTD (22.5, 7.5, and 6 mg/kg/dose, respectively)—Subsequently referred to as the “75% HA triple” arm

Each arm included 10 mice. Drug administration occurred once weekly (all drugs given at the same treatment session) for three weeks. Standard drugs were obtained from the pharmacy and stored as directed: 1 mg/mL cisplatin, mg/mL doxorubicin, and 10 mg/ml, docetaxel. These drugs were diluted in 1×PBS for dosing purposes to the appropriate dosing levels as necessary. HA conjugation was completed for each drug, and HA-doxorubicin and HA-cisplatin were resuspended in water at 1.229 mg/mL and 0.83 mg/mL, respectively, and protected from light at 4° C. HA-doxorubicin was diluted in water as necessary for dosing purposes. Given the limited stability of HA-docetaxel in solution for extended periods of time, solid HA-docetaxel powder was resuspended in water and warmed into solution at 2.8-5.6 mg/mL for each closing session. Standard drugs were injected intraperitoneally at 100 uL total volume per drug. Each HA-conjugated drug was injected at a volume of 100 uL subcutaneously (and peritumorally) about 1-2 mm from the tumor site at different locations around the tumor perimeter.

Following the three cycles of weekly of dosing, two mice were randomly selected from each treatment group and euthanized for immediate post-treatment histological analysis. Samples collected for paraffin blocking included: tumor, brain, heart, lungs, liver, kidneys, spleen, stomach, intestine (ileo-cecal junction), femurs, and skin, and underlying muscle at injection site. Other samples collected (when possible) for freezing included: tumor, urine, and blood serum. Mice reaching morbidity or endpoint criteria during dosing or at any point afterward were similarly euthanized with samples collected when possible. Survival at 12 weeks post-initial dosing was considered the study endpoint.

The study remains ongoing. However, several trends have emerged. First, systemic toxicity as determined by mouse weight, condition monitoring, and mortality was reduced in both the 50% MTD and 75% MTD HA-conjugated triple therapy arms compared to their dose-matched standard therapy groups. 9 of 10 mice in the 75% triple standard arm died or were deemed moribund for euthanasia within 2 weeks of the completion of treatment from toxicity-associated adverse effects. The remaining mouse was euthanized 2 months following the completion of treatment as a result of toxicity-assessed morbidity. 8 of 10 mice in the 50% triple standard-drug arm died or were deemed moribund for euthanasia from toxicity-associated effects as well, 5 within two weeks of the completion of treatment. No mice have been euthanized for toxicity-associated morbidity in either HA triple therapy arm to this point.

Mild to moderate ulceration (grade 1-2 and lasting less than 2 weeks) and fibrosis was observed in the injection regions of the HA triple therapy arms, particularly the 75% MTD group, but did not result in any lasting morbidity. It is hypothesized that this was due to the volume of drug being given in a small subcutaneous space and that this would be reduced or eliminated with injections deeper than the subcutaneous level (i.e., intramuscular) or directly into the tumor. This was not observed with the standard therapies since these were given by a systemic route.

Tumor response to therapy was comparable between the 50% triple standard and 50% HA-conjugated triple arms. Tumor response was significantly improved in the 75% MTD HA-conjugated triple therapy arm compared to the 75% triple therapy standard drug arm. Both HA triple-drug arms (50% MTD and 75% MTD) have included several long-term complete responders (100% tumor response with no residual chemotherapeutic cure). Extended tracking of tumor sizes was limited in the standard therapy groups given early toxicity-associated mortality but no significant tumor reductions were observed in the period the animals remained alive. Control arm tumors were significantly larger than any treatment arm, but only one control animal became moribund during the study period.

### TABLES

Table 1 shows the IC₅₀ values of cisplatin on human breast cancer cell lines MDA-MB-468LN, MDA-MB-231, and MCF-7. IC₅₀s for the HA carriers are given on a Pt-basis equivalent to cisplatin.

TABLE 1

| Cell lines/ | | HA-Cisplatin | HA-Cisplatin |
|---|---|---|---|
| IC₅₀ μg/mL (μM) | Cisplatin | (without silver) | (with silver) |
| MDA-MB-468LN | 5 μg/mL | 10 μg/mL | 9 μg/mL |
| | (17 μM) | (33 μM) | (30 μM) |
| MDA-MB-231 | 6 μg/mL | 10 μg/mL | 4 μg/mL |
| | (20 μM) | (33 μM) | (13 μM) |
| MCF-7 | 6 μg/mL | 11 μg/mL | 6 μg/mL |
| | (20 μM) | (37 μM) | (20 μM) |

Table 2 provides the tissue AUC (average±SEM) and Cₘₐₓ (average±SEM) of 3.3 mg/kg intravenous cisplatin and subcutaneous HA-cisplatin study groups. Two-way ANOVA analysis revealed study groups (cisplatin and HA-cisplatin) differed significantly for all tissues. The first plasma sampling was a 5 minutes.

TABLE 2

| | AUC_{0-96 hrs}, μg/g · h | | Cₘₐₓ, μg/g (Tₘₐₓ) | |
|---|---|---|---|---|
| Tissue | cisplatin, i.v. | HA-cisplatin, s.q. | cisplatin, i.v.* | HA-cisplatin, s.q. |
| Heart | 128 ± 8 | 465 ± 23 | 1.7 ± 0.1 (1 hr) | 14.7 ± 1.1 (1 hr) |
| Lungs | 139 ± 7 | 347 ± 13 | 2.2 ± 0.1 (1 hr) | 7.5 ± 0.7 (1 hr) |
| Kidneys | 291 ± 8 | 669 ± 28 | 4.6 ± 0.4 (1 hr) | 12.3 ± 2.5 (4 hrs) |
| Brain | 152 ± 11 | 344 ± 22 | 2.3 ± 0.1 (1 hr) | 7.5 ± 2.0 (4 hrs) |
| Liver | 178 ± 5 | 495 ± 20 | 2.9 ± 0.2 (4 hrs) | 10.6 ± 2.2 (4 hrs) |
| Spleen | 201 ± 7 | 384 ± 15 | 3.0 ± 0.4 (1 hr) | 6.6 ± 0.6 (4 hrs) |
| Muscle | 151 ± 9 | 262 ± 15 | 2.2 ± 0.2 (1 hr) | 8.5 ± 2.1 (4 hrs) |
| Bladder | 162 ± 10 | 194 ± 6 | 3.1 ± 0.4 (1 hr) | 2.9 ± 0.4 (4 hrs) |
| Ipsilateral axilla | 205 ± 12 | 776 ± 9 | 3.3 ± 0.3 (1 hr) | 20.4 ± 1.4 (1 hr) |
| nodes | | | | |
| Contralateral | 205 ± 12 | 413 ± 17 | 3.3 ± 0.3 (1 hr) | 4.6 ± 0.3 (4 hrs) |
| axilla nodes | | | | |
| Plasma | 17 ± 3 | 67 ± 10 | 3.1 ± 0.2 (5 mins) | 1.0 ± 0.3 (2 hrs) |

*The first tissue sampling was at 1 hour so a Cₘₐₓ prior to this would not be detected.

Table 3 shows the classification of tissue damage for each treatment group was made according to the following scale: Kidneys, grade 0: normal (no symptoms); grade 1: minimal necrosis; grade 2: mild necrosis (includes degeneration and nuclear pyknosis). Liver, grade 0: normal (no symptoms); grade 1: inflammation (includes granulomas, microgranulomas, and hepatitis) or inclusions; grade 2: degeneration or necrosis. Axilla lymph nodes, grade 0: normal (no symptoms); grade 1: mild lymphoid hyperplasia or depletion. Pathologies were graded by a blinded veterinarian pathologist, and each treatment group contained 5 animals.

TABLE 3

| Treatment group | Liver | Kidneys | Axilla lymph nodes |
|---|---|---|---|
| 1.0 mg/kg cisplatin | 40% Grade 0 | 20% Grade 0 | 100% Grade 0 |
| | 40% Grade 1 | 20% Grade 1 | |
| | 20% Grade 2 | 60% Grade 2 | |
| 1.0 mg/kg HA-Pt | 20% Grade 0 | 20% Grade 0 | 60% Grade 0 |
| (Pt refers to cisplatin) | 60% Grade 1 | 80% Grade 1 | 40% Grade 1 |
| | 20% Grade 2 | | |
| 3.3 mg/kg cisplatin | 60% Grade 1 | 20% Grade 1 | 80% Grade 0 |
| | 40% Grade 2 | 80% Grade 2 | 20% Grade 1 |
| 3.3 mg/kg HA-Pt | 20% Grade 0 | 20% Grade 0 | 60% Grade 0 |
| | 60% Grade 1 | 60% Grade 1 | 40% Grade 1 |
| | 20% Grade 2 | 20% Grade 2 | |

Table 4 shows the conjugation efficiency of cisplatin to HA. Efficiency was calculated as (cisplatin added/cisplatin incorporated)×100%.

TABLE 4

| Cisplatin added | Conjugated | Conjugation |
|---|---|---|
| w/w cisplatin/HA | w/w cisplatin/HA | Efficiency, % |
| 0.03 | 0.022 | 73% |
| 0.08 | 0.040 | 50% |
| 0.15 | 0.086 | 57% |
| 0.20 | 0.119 | 60% |
| 0.30 | 0.149 | 50% |
| 0.40 | 0.210 | 53% |
| 0.50 | 0.254 | 51% |
| 0.60 | 0.263 | 44% |
| 0.70 | 0.241 | 34% |

Table 5 shows the tissue AUC of cisplatin and cisplatin-HA. Area-under-the-curve (AUC) of cisplatin after subcutaneous administration of cisplatin or cisplatin-HA into the right mammary fatpad of female rats.

TABLE 5

AUC(0-96 hrs), μg · hr/g

| Tissue | Cisplatin | Cisplatin-HA |
|---|---|---|
| Bladder | 208.5 | 194.5 |
| Brain | 442.5 | 343.7 |
| Heart | 459.0 | 465.3 |
| Kidneys | 650.9 | 668.6 |
| Liver | 415.6 | 495.0 |
| Lungs | 409.8 | 347.3 |
| Muscle | 371.5 | 262.1 |
| Spleen | 349.1 | 383.4 |
| Left lymph node (LLN) | 349.5 | 413.4 |
| Right lymph node (RLN) | 446.0^{†} | 776.0^{†} |
| Plasma | 174.1 | 186.1 |

^{†}p < 0.05.

TABLE 6

| | Release half life, | Release half life, |
|---|---|---|
| Drug/Carrier | (PBS), hrs | (H₂O), hrs |
| Cisplatin-HA | 10 | 42 |
| Cisplatin control | 0.6 | 0.9 |

TABLE 7

| Drug Carrier | IC₅₀ Cisplatin | IC₅₀ Cisplatin-HA |
|---|---|---|
| MDA-MB-468LN | 3 μg/ml* | 3 μg/ml* |
| | (10 μM) | (10 μM) |
| MDA-MB-231 | 4 μg/ml* | 7 μg/ml* |
| | (13 μM) | (23 μM) |
| MCF-7 | 7 μg/ml* | 7 μg/ml* |
| | (23 μM) | (23 μM) |

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

From the foregoing; it will be seen that this invention is one well adapted to attain all ends and objectives herein above set forth, together with the other advantages which are obvious and which are inherent to the invention. Since many possible embodiments may be made of the invention without departing from the scope thereof, it is to be understood that all matters herein set forth or shown in the accompanying figures are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A chemotherapeutic composition configured for administration, the composition comprising:
a pharmaceutically acceptable carrier; and
a nanoconjugate configured for preferential intralymphatic accumulation after percutaneous, intradermal, mucosal, submucosal, subcutaneous, interstitial, intrafat, peritumoral, or intramuscular injection administration, the nanoconjugate having a dimension between 10 and 100 nm; the nanoconjugate comprising:
a nanocarrier configured for preferential intralymphatic accumulation after administration, wherein the nanocarrier is hyaluronan;
a plurality of chemotherapeutic agents coupled to the nanocarrier, wherein the plurality of chemotherapeutic agents comprises doxorubicin.

2. The chemotherapeutic composition as in claim 1 wherein the nanoconjugate has a dimension of about 20 nm to about 80 nm.

3. The chemotherapeutic composition as in claim 1, wherein the nanocarrier is loaded with the chemotherapeutic agents from about 10% to about 50% w/w.

4. The chemotherapeutic composition as in claim 1, wherein the nanocarrier is loaded with the chemotherapeutic agents from about 15% to about 50% w/w.

5. The chemotherapeutic composition as in claim 1, wherein the nanocarrier is loaded with the chemotherapeutic agents from about 25% to about 50% w/w.

6. The chemotherapeutic composition as in claim 1, wherein the nanocarrier is a hyaluronan polymer of about 3 kDa to about 50 kDa.

7. The chemotherapeutic composition as in claim 1, wherein the composition further comprises a second chemotherapeutic agent.

8. The chemotherapeutic composition as in claim 7, wherein said second chemotherapeutic agent is selected from the group consisting of cisplatin, melphalan, mytomycin C, epirubicin, docetaxel, daunorubicin, peptides, interferon, nitrogen mustard class drugs, chlorambucil, amiodarone, topotecan, withaferin A, HSP90 inhibitors, 17-AAG, VEGF inhibitors, histone deacetylase inhibitors, taxanes, taxol, paclitaxel, docetaxel, or combinations thereof.

9. The chemotherapeutic composition as in claim 7, wherein said second chemotherapeutic agent is coupled to a nanoconjugate of hyaluronan.

10. The chemotherapeutic composition as in claim 9, wherein said second chemotherapeutic agent comprises cisplatin.

11. The chemotherapeutic composition as in claim 9, wherein the composition comprises about 50 to 90% of the maximum tolerated dose of cisplatin and about 50 to 90% of the maximum tolerated dose of doxorubicin.

12. The chemotherapeutic composition as in claim 10, wherein said cisplatin is coupled to hyaluronan by mixing the hyaluronan and the cisplatin in an aqueous-based solution.

13. The chemotherapeutic composition as in claim 1, wherein the plurality of chemotherapeutic agents comprising doxorubicin are coupled to the nanocarrier via a biodegradable linker.

14. The chemotherapeutic composition as in claim 13, wherein the biodegradable linker is acid-labile.

15. The chemotherapeutic composition as in claim 14, wherein the biodegradable linker is a dihydrazide.

16. The chemotherapeutic composition as in claim 1, wherein the composition is substantially devoid of PEG, HPMA, polyglutames, and silver.

17. The chemotherapeutic composition as in claim 1, wherein the chemotherapeutic agent is present in a therapeutically effective amount so as to provide a higher lymphatic AUC and a lower plasma Cₘₐₓ compared to standard intravenous administration of the chemotherapeutic agent.

18. The chemotherapeutic composition as in claim 1, wherein the composition further comprises docetaxel coupled to a hyaluronan nanocarrier to form a docetaxel-hyaluronan nanoconjugate having a dimension between about 10 and 100 nm.

19. The chemotherapeutic composition as in claim 18 wherein the composition further comprises cisplatin coupled to a hyaluronan nanocarrier to form a cisplatin-hyaluronan nanoconjugate having a dimension between about 10 and 100 nm.

20. The chemotherapeutic composition as in claim 19 wherein the composition comprises about 50 to 90% of the maximum tolerated dose of cisplatin and about 50 to 90% of the maximum tolerated dose of doxorubicin and 50 to 90% of the maximum tolerated dose of docetaxel.

21. A method for treating and/or inhibiting cancer, the method comprising:
administering the composition of claim 1 to a patient in need thereof.

22. The method as in claim 21, wherein the nanoconjugate has a dimension of about 20 nm to about 80 nm.

23. The method as in claim 21, wherein the nanocarrier is loaded with the chemotherapeutic agents from about 10% to about 50% w/w.

24. The method as in claim 21, wherein the nanocarrier is loaded with the chemotherapeutic agents from about 15% to about 50% w/w.

25. The method as in claim 21, wherein the nanocarrier is loaded with the chemotherapeutic agents from about 25% to about 50% w/w.

26. The method as in claim 21, wherein the nanocarrier is a hyaluronan polymer of about 3 kDa to about 50 kDa.

27. The method as in claim 21, further comprising the step of administering a second chemotherapeutic agent to said patient.

28. The method as in claim 27, wherein said second chemotherapeutic agent is selected from the group consisting of cisplatin, melphalan, mytomycin C, epirubicin, docetaxel, daunorubicin, peptides, interferon, nitrogen mustard class drugs, chlorambucil, amiodarone, topotecan, withaferin A, HSP90 inhibitors, 17-AAG, VEGF inhibitors, histone deacetylase inhibitors, taxanes, taxol, paclitaxel, docetaxel, or combinations thereof.

29. The method as in claim 27, wherein said second chemotherapeutic agent is coupled to a nanoconjugate of hyaluronan.

30. The method as in claim 29, wherein said second chemotherapeutic agent comprises cisplatin.

31. The method as in claim 30 wherein said cisplatin is coupled to hyaluronan by is mixing cisplatin with hyaluronan in an aqueous-based solution.

32. The method as in claim 21, wherein the plurality of chemotherapeutic agents comprising doxorubicin are coupled to the nanocarrier via a biodegradable linker.

33. The method as in claim 32, wherein the biodegradable linker is a dihydrazide.

34. The method as in claim 21, wherein the composition is substantially devoid of PEG, HPMA, polyglutames, and silver.

35. The method as in claim 21, wherein the chemotherapeutic agent is present in a therapeutically effective amount so as to provide a higher lymphatic AUC and a lower plasma Cₘₐₓ compared to standard intravenous administration of the chemotherapeutic agent.

36. The method as in claim 21 wherein said administering step comprises percutaneous administration.

37. The method as in claim 21 wherein said administering step comprises intradermal administration.

38. The method as in claim 21 wherein said administering step comprises subcutaneous administration.

39. The method as in claim 21 wherein said administering step comprises intramuscular injection.

40. The method as in claim 21 further comprising the step of administering a docetaxel-hyaluronan nanoconjugate having a dimension between about 10 and 100 nm.

41. The method as in claim 40 further comprising the step of administering a cisplatin-hyaluronan nanoconjugate having a dimension between about 10 and 100 nm.

42. The method as in claim 41 wherein said administering step comprises administering about 50 to 90% of the maximum tolerated dose of cisplatin and about 50 to 90% of the maximum tolerated dose of doxorubicin and 50 to 90% of the maximum tolerated dose of docetaxel.
